(19) United States
(12) Patent Application Publication
(10) Pub. No.: US 2024/0148445 A1
(43) Pub. Date: May 9, 2024

(54) IMAGE GUIDANCE FOR MEDICAL PROCEDURES

(71) Applicant: Pulmera, Inc., Redwood City, CA (US)

(72) Inventors: Bryan I. Hartley, Nashville, TN (US); Rene Vargas-Voracek, Sunnyvale, CA (US)

(21) Appl. No.: 18/417,589

(22) Filed: Jan. 19, 2024

Related U.S. Application Data
(63) Continuation of application No. PCT/US2022/073876, filed on Jul. 19, 2022.
(60) Provisional application No. 63/261,187, filed on Sep. 14, 2021.
(60) Provisional application No. 63/203,389, filed on Jul. 20, 2021.

(51) Int. Cl. A61B 34/20; A61B 34/10; A61B 34/30; A61B 90/00

(52) U.S. Cl. CPC A61B 34/20; A61B 34/10; A61B 34/30; A61B 2034/105; A61B 2034/2048; A61B 2034/2059; A61B 2034/301; A61B 2034/305; A61B 2090/376; A61B 2560/0223

(57) ABSTRACT

Systems, methods, and devices for medical imaging are disclosed herein. In some embodiments, a system for imaging an anatomic region includes one or more processors, a display, and a memory storing instructions that, when executed by the one or more processors, cause the system to perform various operations. The operations can include generating a 3D reconstruction of an anatomic region from first image data obtained using an imaging apparatus, and identifying a target structure in the 3D reconstruction. The operations can also include receiving second image data of the anatomic region obtained using the imaging apparatus, and receiving pose data of an imaging arm of the imaging apparatus. The operations can further include outputting, via the display, a graphical representation of the target structure overlaid onto the second image data, based on the pose data and the 3D reconstruction.

### CROSS-REFERENCE TO RELATED APPLICATION(S)

The present application is a continuation of International Application No. PCT/US2022/073876, filed Jul. 19, 2022, which claims the benefit of priority to U.S. Provisional Application No. 63/203,389, filed Jul. 20, 2021; and U.S. Provisional Application No. 63/261,187, filed Sep. 14, 2021; each of which is incorporated by reference herein in its entirety.

This application is related to U.S. patent application Ser. No. 17/658,642, filed Apr. 8, 2022, entitled “MEDICAL IMAGING SYSTEMS AND ASSOCIATED DEVICES AND METHODS,” which is incorporated by reference herein in its entirety.

### TECHNICAL FIELD

The present technology relates generally to medical imaging, and in particular, to methods for providing image guidance for medical procedures.

### BACKGROUND

3D anatomic models, such as computed tomography (CT) volumetric reconstructions, are frequently used in image-guided medical procedures to allow the physician to visualize the patient anatomy in three dimensions and accurately position surgical tools at the appropriate locations. However, 3D models generated from preprocedural image data may not accurately reflect the actual anatomy at the time of the procedure. Moreover, if the model is not correctly registered to the anatomy, it may be difficult or impossible for the physician to navigate the tool to the right location, thus compromising the accuracy and efficacy of the procedure.

Cone-beam computed tomography (CBCT) has been used to generate high resolution, 3D volumetric reconstructions of a patient's anatomy for image guidance during a medical procedure. However, many physicians do not have ready access to conventional CBCT imaging systems because these systems are extremely expensive and often reserved for use by specialty departments. While tomosynthesis (also known as limited-angle tomography) has also been used for intraprocedural imaging, this technique is unable to produce 3D reconstructions with sufficiently high resolution for many procedures. Accordingly, improved medical imaging systems and methods are needed.

### BRIEF DESCRIPTION OF THE DRAWINGS

Many aspects of the present disclosure can be better understood with reference to the following drawings. The components in the drawings are not necessarily to scale. Instead, emphasis is placed on clearly illustrating the principles of the present disclosure.

FIGS. 1A-1D illustrate a system for imaging a patient, in accordance with embodiments of the present technology.

FIG. 2 is a flow diagram illustrating a method for imaging an anatomic region, in accordance with embodiments of the present technology.

FIG. 3A is a flow diagram illustrating a method for imaging an anatomic region, in accordance with embodiments of the present technology.

FIG. 3B is a representative example of an augmented fluoroscopic image, in accordance with embodiments of the present technology.

FIG. 4 is a flow diagram illustrating a method for imaging an anatomic region, in accordance with embodiments of the present technology.

FIG. 5 is a flow diagram illustrating a method for imaging an anatomic region during a treatment procedure, in accordance with embodiments of the present technology.

FIG. 6A illustrates a tool positioned within a target structure, in accordance with embodiments of the present technology.

FIG. 6B illustrates the tool and target structure of FIG. 6A after a treatment procedure.

FIG. 6C illustrates a subtraction image generated from pre- and post-treatment images of the target structure of FIGS. 6A and 6B.

FIGS. 7A and 7B illustrate an imaging apparatus and a robotic assembly, in accordance with embodiments of the present technology.

FIG. 8 is a flow diagram illustrating a method for imaging an anatomic region in combination with a robotic assembly, in accordance with embodiments of the present technology.

FIG. 9 is a flow diagram illustrating a method for imaging an anatomic region, in accordance with embodiments of the present technology.

FIG. 10 is a flow diagram illustrating a method for aligning an imaging apparatus with a target structure, in accordance with embodiments of the present technology.

FIG. 11 is a flow diagram illustrating a method for using an imaging apparatus in combination with a robotic assembly, in accordance with embodiments of the present technology.

### DETAILED DESCRIPTION

The present technology generally relates to systems, methods, and devices for medical imaging. For example, in some embodiments, the systems and methods described herein use a mobile C-arm x-ray imaging apparatus (also referred to herein as a “mobile C-arm apparatus”) to generate a 3D reconstruction of a patient's anatomy using CBCT imaging techniques. Unlike conventional systems and devices that are specialized for CBCT imaging, the mobile C-arm apparatus may lack a motor and/or other automated mechanisms for rotating the imaging arm that carries the x-ray source and detector. Instead, the imaging arm is manually rotated through a series of different angles to obtain a sequence of two-dimensional (2D) projection images of the anatomy.

In some embodiments, the present technology provides methods for imaging an anatomic region using a manually-operated imaging apparatus such as a mobile C-arm apparatus. The method can include generating a 3D reconstruction of the anatomic region using the imaging apparatus. The 3D reconstruction can be generated from images acquired by the imaging apparatus during a manual rotation, as well as pose data of the imaging apparatus during the rotation. The 3D reconstruction can be used to provide image-based guidance to an operator in various medical procedures. For example, the 3D reconstruction can be used to augment or otherwise annotate live image data (e.g., fluoroscopic data) with relevant information for the procedure, such as the location of a target structure to be biopsied, treated, etc. As another example, the 3D reconstruction can also be used to update, correct, or otherwise modify a registration between a medical instrument and a preoperative anatomic model. In a further example, multiple 3D reconstructions can be generated before and after treating (e.g., ablating) a target structure. The 3D reconstructions before and after treatment can be compared in order to determine changes in the target after treatment.

The present technology also provides methods for operating an imaging apparatus in combination with a robotic system, such as a robotic assembly or platform for navigating a medical or surgical tool (e.g., an endoscope, biopsy needle, ablation probe, etc.) within the patient's anatomy. The presence of the robotic assembly may constrain the rotational range of the imaging apparatus. Accordingly, the present technology can provide methods for adapting the imaging techniques described herein for use with a robotic assembly. For example, in some embodiments, a method for imaging an anatomic region includes positioning a tool at a target location in the anatomic region using the robotic assembly. The tool can then be disconnected from the robotic assembly. A manually-operated imaging apparatus can be used to generate a 3D reconstruction of the anatomic region while the robotic assembly is disconnected. The tool can then be reconnected to the robotic assembly and registered to the target location. As another example, a method for imaging an anatomic region can include obtaining first image data over a larger angular range before the robotic assembly is positioned near the patient, and obtaining second image data over a smaller angular range after the robotic assembly is positioned near the patient. The first and second image data can be combined and used to generate a 3D reconstruction that is displayed to provide intraprocedural guidance to the operator.

The embodiments described herein can provide many advantages over conventional imaging technologies. For example, the systems and methods herein can use a manually-rotated mobile C-arm apparatus to generate high quality CBCT images of a patient's anatomy, rather than a specialized CBCT imaging system. This approach can reduce costs and increase the availability of CBCT imaging, thus allowing CBCT imaging techniques to be used in many different types of medical procedures. For example, CBCT imaging can be used to generate intraprocedural 3D models of an anatomic region for guiding a physician in many types of medical procedures, such as a biopsy procedure, ablation procedure, or other diagnostic or treatment procedures (e.g., lung procedures, orthopedic procedures, etc.). Additionally, the techniques described herein allow CBCT imaging to be used in combination with robotically-controlled medical or surgical systems, thus enhancing the accuracy and efficiency of procedures performed with such systems.

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

As used herein, the terms “vertical,” “lateral,” “upper,” and “lower” can refer to relative directions or positions of features of the embodiments disclosed herein in view of the orientation shown in the Figures. For example, “upper” or “uppermost” can refer to a feature positioned closer to the top of a page than another feature. These terms, however, should be construed broadly to include embodiments having other orientations, such as inverted or inclined orientations where top/bottom, over/under, above/below, up/down, and left/right can be interchanged depending on the orientation.

Although certain embodiments of the present technology are described in the context of medical procedures performed in the lungs, this is not intended to be limiting. Any of the embodiments disclosed herein can be used in other types of medical procedures, such as procedures performed on or in the musculoskeletal system, vasculature, abdominal cavity, gastrointestinal tract, genitourinary tract, brain, and so on. Additionally, any of the embodiments herein can be used for applications such as surgical tool guidance, biopsy, ablation, chemotherapy administration, surgery, or any other procedure for diagnosing or treating a patient.

The headings provided herein are for convenience only and do not interpret the scope or meaning of the claimed present technology.

### I. Overview of Technology

Lung cancer kills more people each year than breast, prostate, and colon cancers combined. Most lung cancers are diagnosed at a late stage, which contributes to the high mortality rate. Earlier diagnosis of lung cancer (e.g., at stages 1-2) can greatly improve survival. The first step in diagnosing an early-stage lung cancer is to perform a lung biopsy on the suspicious nodule or lesion. Bronchoscopic lung biopsy is the conventional biopsy route, but typically suffers from poor success rates (e.g., only 50% to 70% of nodules are correctly diagnosed), meaning that the cancer status of many patients remains uncertain even after the biopsy procedure. One common reason for non-diagnostic biopsy is that the physician fails to place the biopsy needle into the correct location in the nodule before collecting the biopsy sample. This situation can occur due to shortcomings of conventional technologies for guiding the physician in navigating the needle to the target nodule. For example, conventional technologies typically use a static chest CT scan of the patient obtained before the biopsy procedure (e.g., days to weeks beforehand) that is registered to the patient's anatomy during the procedure (e.g., via electromagnetic (EM) navigation or shape sensing technologies). Registration errors can cause the physician to completely miss the nodule during needle placement. These errors, also known as CT-to-body divergence, occur when the preprocedural scan data does not match the patient anatomy data obtained during the actual procedure. These differences can occur because the lungs are dynamic and often change in volume from day-to-day and/or when patients are under anesthesia. Research has shown that the average error between the preprocedural CT scan and the patient's anatomy during the procedure is 1.8 cm, which is larger than many of the pulmonary nodules being biopsied.

CBCT is an imaging technique capable of producing high resolution 3D volumetric reconstructions of a patient's anatomy. For bronchoscopic lung biopsy, intraprocedural CBCT imaging can be used to confirm that the biopsy needle is positioned appropriately relative to the target nodule and has been shown to increase diagnostic accuracy by almost 20%. A typical CBCT procedure involves scanning the patient's body with a cone-shaped x-ray beam that is rotated over a wide, circular arc (e.g., 180° to 360°) to obtain a sequence of 2D projection images. A 3D volumetric reconstruction of the anatomy can be generated from the 2D images using image reconstruction techniques such as filtered backprojection or iterative reconstruction. Conventional CBCT imaging systems include a motorized imaging arm for automated, highly-controlled rotation of the x-ray source and detector over a smooth, circular arc during image acquisition. These systems are also capable of accurately tracking the pose of the imaging arm across different rotation angles. However, CBCT imaging systems are typically large, extremely expensive, and may not be available to many physicians, such as pulmonologists performing lung biopsy procedures.

Tomosynthesis is a technique that may be used to generate intraprocedural images of patient anatomy. However, because tomosynthesis uses a much smaller rotation angle during image acquisition (e.g., 15° to 70°), the resulting images are typically low resolution, lack sufficient depth information, and/or may include significant distortion. Tomosynthesis is therefore typically not suitable for applications requiring highly accurate 3D spatial information.

Accordingly, there is a need for imaging techniques that are capable of producing intraprocedural, high resolution 3D representations of a patient's anatomy using low-cost, accessible imaging systems. such as mobile C-arm apparatuses. The present technology can address these and other challenges by providing systems, methods, and devices for performing CBCT imaging using a manually-rotated imaging apparatus, also referred to herein as “manually-rotated CBCT” or “mrCBCT.” Manually-operated imaging apparatus such as mobile C-arm apparatuses are generally less expensive and more readily available than specialized CBCT imaging systems, and can be adapted for mrCBCT imaging using the stabilization and calibration techniques described herein. The systems, methods, and devices disclosed herein can be used to assist an operator in performing a medical procedure, such as by providing image-based guidance based on mrCBCT images and/or by adapting mrCBCT imaging techniques for use with robotically-controlled systems.

### II. Medical Imaging Systems and Associated Devices and Methods

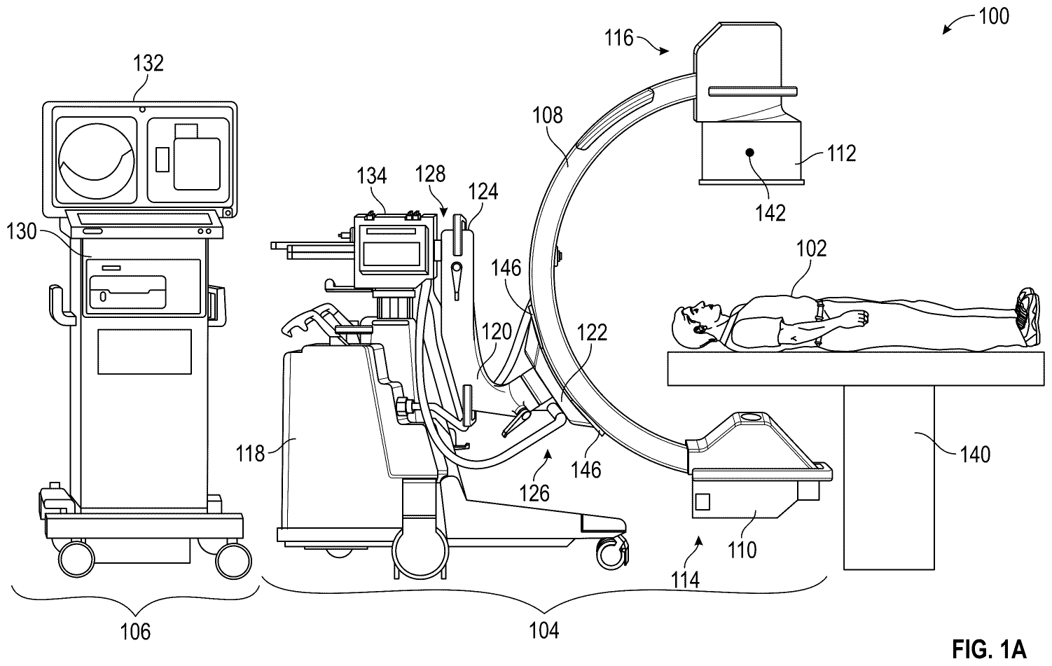

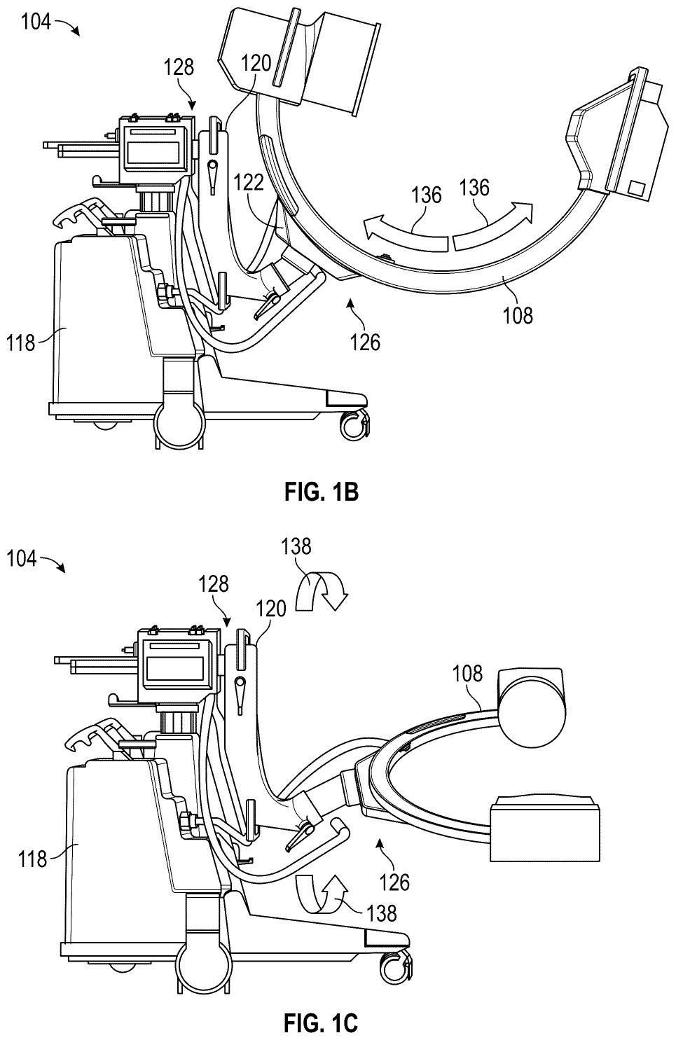

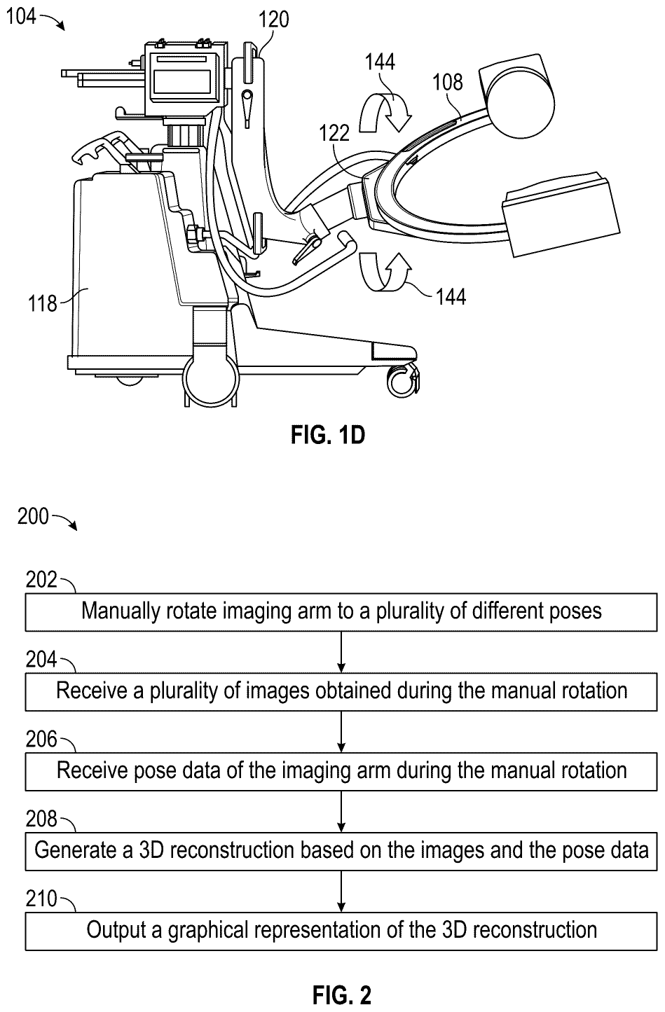

FIG. 1A is a partially schematic illustration of a system 100 for imaging a patient 102 in accordance with embodiments of the present technology. The system 100 includes an imaging apparatus 104 operably coupled to a console 106. The imaging apparatus 104 can be any suitable device configured to generate images of a target anatomic region of the patient 102, such as an x-ray imaging apparatus. In the illustrated embodiment, for example, the imaging apparatus 104 is a mobile C-arm apparatus configured for fluoroscopic imaging. A mobile C-arm apparatus typically includes a manually-movable imaging arm 108 configured as a curved, C-shaped gantry (also known as a “C-arm”). Examples of mobile C-arm apparatuses include, but are not limited to, the OEC 9900 Elite (GE Healthcare) and the BV Pulsera (Philips). In other embodiments, however, the techniques described herein can be adapted to other types of imaging apparatuses 104 having a manually-movable imaging arm 108, such as a G-arm imaging apparatus.

The imaging arm 108 can carry a radiation source 110 (e.g., an x-ray source) and a detector 112 (e.g., an x-ray detector such as an image intensifier or flat panel detector). The radiation source 110 can be mounted at a first end portion 114 of the imaging arm 108, and the detector 112 can be mounted at a second end portion 116 of the imaging arm 108 opposite the first end portion 114. During a medical procedure, the imaging arm 108 can be positioned near the patient 102 such that the target anatomic region is located between the radiation source 110 and the detector 112. The imaging arm 108 can be rotated to a desired pose (e.g., angle) relative to the target anatomic region. The radiation source 110 can output radiation (e.g., x-rays) that travels through the patient's body to the detector 112 to generate 2D images of the anatomic region (also referred to herein as “projection images”). The image data can be output as still or video images. In some embodiments, the imaging arm 108 is rotated through a sequence of different poses to obtain a plurality of 2D projection images. The images can be used to generate a 3D representation of the anatomic region (also referred to herein as a “3D reconstruction,” “volumetric reconstruction,” “image reconstruction,” or “CBCT reconstruction”). The 3D representation can be displayed as a 3D model or rendering, and/or as one or more 2D image slices (also referred to herein as “CBCT images” or “reconstructed images”).

In some embodiments, the imaging arm 108 is coupled to a base 118 by a support arm 120. The base 118 can act as a counterbalance for the imaging arm 108, the radiation source 110, and the detector 112. As shown in FIG. 1A, the base 118 can be a mobile structure including wheels for positioning the imaging apparatus 104 at various locations relative to the patient 102. In other embodiments, however, the base 118 can be a stationary structure. The base 118 can also carry various functional components for receiving, storing, and/or processing the image data from the detector 112, as discussed further below.

The support arm 120 (also referred to as an “attachment arm” or “pivot arm”) can connect the imaging arm 108 to the base 118. The support arm 120 can be an elongate structure having a distal portion 122 coupled to the imaging arm 108, and a proximal portion 124 coupled to the base 118. Although the support arm 120 is depicted in FIG. 1A as being an L-shaped structure (“L-arm”) having a vertical section and a horizontal section, in other embodiments the support arm 120 can have a different shape (e.g., a curved shape).

The imaging arm 108 can be configured to rotate in multiple directions relative to the base 118. For example, FIG. 1B is a partially schematic illustration of the imaging apparatus 104 during an orbital rotation. As shown in FIG. 1B, during an orbital rotation, the imaging arm 108 rotates relative to the support arm 120 and base 118 along a lengthwise direction as indicated by arrows 136. Thus, during an orbital rotation, the motion trajectory can be located primarily or entirely within the plane of the imaging arm 108. The imaging arm 108 can be slidably coupled to the support arm 120 to allow for orbital rotation of the imaging arm 108. For example, the imaging arm 108 can be connected to the support arm 120 via a first interface 126 that allows the imaging arm 108 to slide along the support arm 120.

FIG. 1C is a partially schematic illustration of the imaging apparatus 104 during a propeller rotation (also known as “angular rotation” or “angulation”). As shown in FIG. 1C, during a propeller rotation, the imaging arm 108 and support arm 120 rotate relative to the base 118 in a lateral direction as indicated by arrows 138. The support arm 120 can be rotatably coupled to the base 118 via a second interface 128 (e.g., a pivoting joint or other rotatable connection) that allows the imaging arm 108 and support arm 120 to turn relative to the base 118. Optionally, the imaging apparatus 104 can include a locking mechanism to prevent orbital rotation while the imaging arm 108 is performing a propeller rotation, and/or to prevent propeller rotation while the imaging arm 108 is performing an orbital rotation.

The imaging apparatus 104 can optionally be configured to rotate in other directions, alternatively or in addition to orbital rotation and/or propeller rotation. For example, FIG. 1D is a partially schematic illustration of the imaging apparatus 104 during a flip-flop rotation. As shown in FIG. 1D, during a flip-flop rotation, the imaging arm 108 and the distal portion 122 of the support arm 120 rotate laterally relative to the rest of the support arm 120 and the base 118, as indicated by arrows 144. A flip-flop rotation may be advantageous in some situations for reducing interference with other components located near the operating table 140 (e.g., a surgical robotic assembly).

Referring again to FIG. 1A, the imaging apparatus 104 can be operably coupled to a console 106 for controlling the operation of the imaging apparatus 104. As shown in FIG. 1A, the console 106 can be a mobile structure with wheels, thus allowing the console 106 to be moved independently of the imaging apparatus 104. In other embodiments, however, the console 106 can be a stationary structure. The console 106 can be attached to the imaging apparatus 104 by wires, cables, etc., or can be a separate structure that communicates with the imaging apparatus 104 via wireless communication techniques. The console 106 can include a computing device 130 (e.g., a workstation, personal computer, laptop computer, etc.) including one or more processors and memory configured to perform various operations related to image acquisition and/or processing. For example, the computing device 130 can perform some or all of the following operations: receive, organize, store, and/or process data (e.g., image data, sensor data, calibration data) relevant to generating a 3D reconstruction; execute image reconstruction algorithms; execute calibration algorithms; and post-process, render, and/or display the 3D reconstruction. Additional examples of operations that may be performed by the computing device 130 are described in greater detail elsewhere herein.

The computing device 130 can receive data from various components of the system 100. For example, the computing device 130 can be operably coupled to the imaging apparatus 104 (e.g., to radiation source 110, detector 112, and/or base 118) via wires and/or wireless communication modalities (e.g., Bluetooth, WiFi) so that the computing device 130 can transmit commands to the imaging apparatus 104 and/or receive data from the imaging apparatus 104. In some embodiments, the computing device 130 transmits commands to the imaging apparatus 104 to cause the imaging apparatus 104 to start acquiring images, stop acquiring images, adjust the image acquisition parameters, and so on. The imaging apparatus 104 can transmit image data (e.g., the projection images acquired by the detector 112) to the computing device 130. The imaging apparatus 104 can also transmit status information to the computing device 130, such as whether the components of the imaging apparatus 104 are functioning properly, whether the imaging apparatus 104 is ready for image acquisition, whether the imaging apparatus 104 is currently acquiring images, etc.

Optionally, the computing device 130 can also receive other types of data from the imaging apparatus 104. In the embodiment of FIG. 1A, for example, the imaging apparatus 104 includes at least one sensor 142 configured to generate sensor data indicative of a pose of the imaging arm 108. The sensor data can be transmitted to the computing device 130 via wired or wireless communication for use in the image processing techniques described herein. Additional details of the configuration and operation of the sensor 142 are provided below.

The console 106 can include various user interface components allowing an operator (e.g., a physician, nurse, technician, or other healthcare professional) to interact with the computing device 130. For example, the operator can input commands to the computing device 130 via a suitable input device (e.g., a keyboard, mouse, joystick, touchscreen, microphone). The console 106 can also include a display 132 (e.g., a monitor or touchscreen) for outputting image data, sensor data, reconstruction data, status information, control information, and/or any other suitable information to the operator. Optionally, the base 118 can also include a secondary display 134 for outputting information to the operator.

Although FIG. 1A shows the console 106 as being separate from the imaging apparatus 104, in other embodiments the console 106 can be physically connected to the imaging apparatus 104 (e.g., to the base 118), such as by wires, cables, etc. Additionally, in other embodiments, the base 118 can include a respective computing device and/or input device, such that the imaging apparatus 104 can also be controlled from the base 118. In such embodiments, the computing device located in the base 118 can be configured to perform any of the image acquisition and/or processing operations described herein. Optionally, the console 106 can be integrated with the base 118 (e.g., the computing device 130 is located in the base 118) or omitted altogether such that the imaging apparatus 104 is controlled entirely from the base 118. In some embodiments, the system 100 includes multiple consoles 106 (e.g., at least two consoles 106), each with a respective computing device 130. Any of the processes described herein can be performed on a single console 106 or across any suitable combination of multiple consoles 106.

In some embodiments, the system 100 is used to perform an imaging procedure in which an operator manually rotates the imaging arm 108 during imaging acquisition, such as an mrCBCT procedure. In such embodiments, the imaging apparatus 104 can be a manually-operated device that lacks any motors or other actuators for automatically rotating the imaging arm 108. For example, one or both of the first interface 126 and second interface 128 can lack any automated mechanism for actuating orbital rotation and propeller rotation of the imaging arm 108, respectively. Instead, the user manually applies the rotational force to the imaging arm 108 and/or support arm 120 during the mrCBCT procedure.

In some embodiments, the imaging procedure involves performing a propeller rotation of the imaging arm 108. Propeller rotation may be advantageous for mrCBCT or other imaging techniques that involve rotating the imaging arm 108 over a relatively large rotation angle. For example, a mrCBCT or similar imaging procedure can involve rotating the imaging arm 108 over a range of at least 90°, 100°, 110°, 120°, 130°, 140°, 150°, 160°, 170°, 180°, 190°, 200°, 210°, 220°, 230°, 240°, 250°, 260°, 270°, 280°, 290°, 300°, 310°, 320°, 330°, 340°, 350°, or 360°. The total rotation can be within a range from 90° to 360°, 90° to 270°, 90° to 180°, 120° to 360°, 120° to 270°, 120° to 180°, 180° to 360°, or 180° to 270°. As previously discussed, the large rotation angle may be helpful or necessary for capturing a sufficient number of images from different angular positions to generate an accurate, high resolution 3D reconstruction of the anatomy.

In some embodiments, the system 100 includes one or more shim structures 146 for mechanically stabilizing certain portions of the imaging apparatus 104 during an mrCBCT procedure (the shim structures 146 are omitted in FIGS. 1B-1D merely for purposes of simplicity). The shim structures 144 can be removable or permanent components that are coupled to the imaging apparatus 104 at one or more locations to reduce or prevent unwanted movements during a manual rotation. In the illustrated embodiment, the system 100 includes two shim structures 146 positioned at opposite ends of the first interface 126 between the imaging arm 108 and the support arm 120. Optionally, the system 100 can include four shim structures 146, one at each end of the first interface 126 and on both lateral sides of the first interface 126. Alternatively or in combination, the system 100 can include one or more shim structures 146 at other locations of the imaging apparatus 104 (e.g., at the second interface 128). Any suitable number of shim structures 146 can be used, such as one, two, three, four, five, six, seven, eight, nine, ten, 11, 12, or more shim structures.

The shim structures 146 can be elongate members, panels, blocks, wedges, etc., configured to fill a space between two or more components of the imaging apparatus 104 (e.g., between the imaging arm 108 and support arm 120) to reduce or prevent those components from moving relative to each other. The shim structures 146 can make it easier for a user to produce a smooth, uniform, and/or reproducible movement of the imaging arm 108 over a wide rotation angle without using motors or other automated actuation mechanisms. Accordingly, the projection images generated by the detector 112 can exhibit little or no bumps or oscillations, thus improving the ability to generate consistent, high quality 3D reconstructions.

Alternatively or in combination, the mechanical stability of the imaging apparatus 104 during manual rotation can be improved by applying force closer to the center of rotation. For example, for a manual propeller rotation, the operator can apply force to the proximal portion 124 of the support arm 120 at or near the second interface 128, rather than to the imaging arm 108. In some embodiments, to reduce the amount of force for performing a manual propeller rotation at or near the second interface 128, the system 100 can include a temporary or permanent lever structure (not shown) that attaches to the proximal portion 124 of the support arm 120 near the second interface 128 to provide greater mechanical advantage for rotation. The lever structure can include a clamp section configured to couple to the support arm 120, and a handle connected to the clamp section. Accordingly, the operator can grip and apply force to the handle in order to rotate the imaging arm 108.

During an mrCBCT procedure, the imaging arm 108 can be rotated to a plurality of different angles while the detector 112 obtains 2D images of the patient's anatomy. In some embodiments, to generate a 3D reconstruction from the 2D images, the pose of the imaging arm 108 needs to be determined for each image with a high degree of accuracy. Accordingly, the system 100 can include at least one sensor 142 for tracking the pose of the imaging arm 108 during a manual rotation. The sensor 142 can be positioned at any suitable location on the imaging apparatus 104. In the illustrated embodiment, for example, the sensor 142 is positioned on the detector 112. Alternatively or in combination, the sensor 142 can be positioned at a different location, such as on the radiation source 110, on the imaging arm 108 (e.g., at or near the first end portion 114, at or near the second end portion 116), on the support arm 120 (e.g., at or near the distal portion 122, at or near the proximal portion 124), and so on. Additionally, although FIG. 1A illustrates a single sensor 142, in other embodiments, the system 100 can include multiple sensors 142 (e.g., two, three, four, five, or more sensors 142) distributed at various locations on the imaging apparatus 104. For example, the system 100 can include a first sensor 142 on the detector 112, a second sensor 142 on the radiation source 110, etc. The sensors 142 can be removably coupled or permanently affixed to the imaging apparatus 104.

The sensor 142 can be any sensor type suitable for tracking the pose (e.g., position and/or orientation) of a movable component. For example, the sensor 142 can be configured to track the rotational angle of the imaging arm 108 during a manual propeller rotation. Examples of sensors 142 suitable for use with the imaging apparatus 104 include, but are not limited to, motion sensors (e.g., IMUs, accelerometers, gyroscopes, magnetometers), light and/or radiation sensors (e.g., photodiodes), image sensors (e.g., video cameras), EM sensors (e.g., EM trackers or navigation systems), shape sensors (e.g., shape sensing fibers or cables), or suitable combinations thereof. In embodiments where the system 100 includes multiple sensors 142, the sensors 142 can be the same or different sensor types. For example, the system 100 can include two motion sensors, a motion sensor and a photodiode, a motion sensor and a shape sensor, etc.

Additional examples and features of shim structures, lever structures, and sensors suitable for use with the system 100 of FIGS. 1A-1D are described in U.S. patent application Ser. No. 17/658,642, filed Apr. 8, 2022, entitled “MEDICAL IMAGING SYSTEMS AND ASSOCIATED DEVICES AND METHODS,” which is incorporated by reference herein in its entirety.

FIG. 2 is a block diagram illustrating a method 200 for imaging an anatomic region, in accordance with embodiments of the present technology. The method 200 can be performed using any embodiment of the systems and devices described herein, such as the system 100 of FIGS. 1A-1D. The method 200 disclosed herein can be performed by an operator (e.g., a physician, nurse, technician, or other healthcare professional), by a computing device (e.g., the computing device 130 of FIG. 1A), or suitable combinations thereof. For example, some processes in the method 200 can be performed manually by an operator, while other processes in the method 200 can be performed automatically or semi-automatically by one or more processors of a computing device.

The method 200 begins at block 202 with manually rotating an imaging arm to a plurality of different poses. The imaging arm can be part of an imaging apparatus, such as the imaging apparatus 104 of FIG. 1A. For example, the imaging apparatus can be a mobile C-arm apparatus, and the imaging arm can be the C-arm of the mobile C-arm apparatus. The imaging arm can be rotated around a target anatomic region of a patient along any suitable direction, such as a propeller rotation direction. In some embodiments, the imaging arm is manually rotated to a plurality of different poses (e.g., angles) relative to the target anatomic region. The imaging arm can be rotated through an arc that is sufficiently large for performing CBCT imaging. For example, the arc can be at least 90°, 100°, 110°, 120°, 130°, 140°, 150°, 160°, 170°, 180°, 190°, 200°, 210°, 220°, 230°, 240°, 250°, 260°, 270°, 280°, 290°, 300°, 310°, 320°, 330°, 340°, 350°, or 360°.

In some embodiments, the imaging apparatus is stabilized to reduce or prevent undesirable movements (e.g., oscillations, jerks, shifts, flexing, etc.) during manual rotation. For example, the imaging arm can be stabilized using one or more shim structures (e.g., the shim structures 146 of FIG. 1A). Alternatively or in combination, the imaging arm can be rotated by applying force to the support arm (e.g., to the proximal portion of the support arm at or near the center rotation), rather than by applying force to the imaging arm. As previously described, the force can be applied via one or more lever structures coupled to the support arm. In other embodiments, however, the imaging arm can be manually rotated without any shim structures and/or without applying force to the support arm.

At block 204, the method 200 continues with receiving a plurality of images obtained during the manual rotation. The images can be 2D projection images generated by a detector (e.g., an image intensifier or flat panel detector) carried by the imaging arm. The method 200 can include generating any suitable number of images, such as at least 50, 100, 200, 300, 400, 500, 600, 700, 800, 900, or 1000 images. The images can be generated at a rate of at least 5 images per second, 10 images per second, 20 images per second, 30 images per second, 40 images per second, 50 images per second, or 60 images per second. In some embodiments, the images are generated while the imaging arm is manually rotated through the plurality of different poses, such that some or all of the images are obtained at different poses of the imaging arm.

At block 206, the method 200 can include receiving pose data of the imaging arm during the manual rotation. The pose data can include data representing the position and/or orientation of the imaging arm, such as the rotational angle of the imaging arm. In some embodiments, the pose data is generated or otherwise determined based on sensor data from at least one sensor (e.g., the sensor 142 of FIG. 1A). The sensor can be an IMU or another motion sensor coupled to the imaging arm (e.g., to the detector), to the support arm, or a combination thereof. The sensor data can be processed to determine the pose of the imaging arm at various times during the manual rotation. In some embodiments, the pose of the imaging arm is estimated without using a fiducial marker board or other reference object positioned near the patient.

At block 208, the method 200 includes generating a 3D reconstruction based on the images received in block 204 and the pose data received in block 206. The 3D reconstruction process can include several steps. For example, the pose data can first be temporally synchronized with the images generated in block 204, such that each image is associated with a corresponding pose (e.g., rotational angle) of the imaging arm at the time the image was obtained. In some embodiments, the pose data and the image data are time stamped, and the method 200 includes comparing the time stamps to determine the pose (e.g., rotational angle) of the imaging arm at the time each image was acquired. The synchronization process can be performed by a controller or other device that is operably coupled to the output from the imaging apparatus and/or the sensor producing the motion data.

Next, one or more distortion correction parameters can be applied to some or all of the images. Distortion correction can be used in situations where the imaging apparatus produces image distortion. For example, in embodiments where the detector is an image intensifier, the resulting images can exhibit pincushion and/or barrel distortion, among others. The distortion correction parameters can be applied to the images to reduce or eliminate the distortion. In some embodiments, the distortion correction parameters are determined in a previous calibration process.

Subsequently, one or more geometric calibration parameters can be applied to some or all of the images. The geometric calibration parameters can be used to reduce or eliminate misalignment between the images, e.g., due to undesirable motions of the imaging apparatus during image acquisition. For example, during a manual rotation, the imaging arm may shift laterally outside of the desired plane of movement and/or may rotate in a non-circular manner. The geometric calibration parameters can adjust the images to compensate for these motions. In some embodiments, the geometric calibration parameters are determined in a previous calibration process.

In some embodiments, the distortion correction parameters and/or geometric calibration parameters can be adjusted to account for any deviations from the calibration setup. For example, if the manual rotation trajectory of the imaging apparatus in block 202 differs significantly from the rotation trajectory used in the previous calibration process, the resulting reconstruction may not be sufficiently accurate if computed using the original distortion correction and/or geometric calibration parameters. Accordingly, the method 200 can include detecting when significant deviations are present (e.g., based on the pose data generated in block 206), and modifying the distortion correction parameters and/or calibration parameters based on the actual trajectory of the imaging apparatus.

The adjusted images and the pose data associated with the images can then be used to generate a 3D reconstruction from the images, in accordance with techniques known to those of skill in the art. For example, the 3D reconstruction can be generated using filtered backprojection, iterative reconstruction, and/or other suitable algorithms.

At block 210, the method 200 can optionally include outputting a graphical representation of the 3D reconstruction. The graphical representation can be displayed on an output device (e.g., the display 132 and/or secondary display 134 of FIG. 1A) to provide guidance to a user in performing a medical procedure. In some embodiments, the graphical representation includes the 3D reconstruction generated in block 208, e.g., presented as a 3D model or other virtual rendering. Alternatively or in combination, the graphical representation can include 2D images derived from the 3D reconstruction (e.g., 2D axial, coronal, and/or sagittal image slices).

In some embodiments, the user views the graphical representation to confirm whether a medical tool is positioned at a target location. For example, the graphical representation can be used to verify whether a biopsy instrument is positioned within a nodule or lesion of interest. As another example, the graphical representation can be used to determine whether an ablation device is positioned at or near the tissue to be ablated. If the tool is positioned properly, the user can proceed with performing the medical procedure. If the graphical representation indicates that the tool is not at the target location, the user can reposition the tool, and then repeat some or all of the processes of the method 200 to generate a new 3D reconstruction of the tool and/or target within the anatomy.

Additional examples and features of imaging and calibration processes that may be used in combination with the method 200 are described in U.S. patent application Ser. No. 17/658,642, filed Apr. 8, 2022, entitled “MEDICAL IMAGING SYSTEMS AND ASSOCIATED DEVICES AND METHODS,” which is incorporated by reference herein in its entirety.

### III. Methods for Imaging and Providing Image Guidance

In some embodiments, the present technology provides methods for imaging an anatomic region of a patient and/or outputting image guidance for a medical procedure, using the mrCBCT approaches described above. Any of the methods disclosed herein can be performed using any embodiment of the systems and devices described herein, such as the system 100 of FIGS. 1A-1D. The methods herein disclosed herein can be performed by an operator (e.g., a physician, nurse, technician, or other healthcare professional), by a computing device (e.g., the computing device 130 of FIG. 1A), or suitable combinations thereof. For example, some processes in the methods herein can be performed manually by an operator, while other processes in the methods herein can be performed automatically or semi-automatically by one or more processors of a computing device. Any of the methods described herein can be combined with each other.

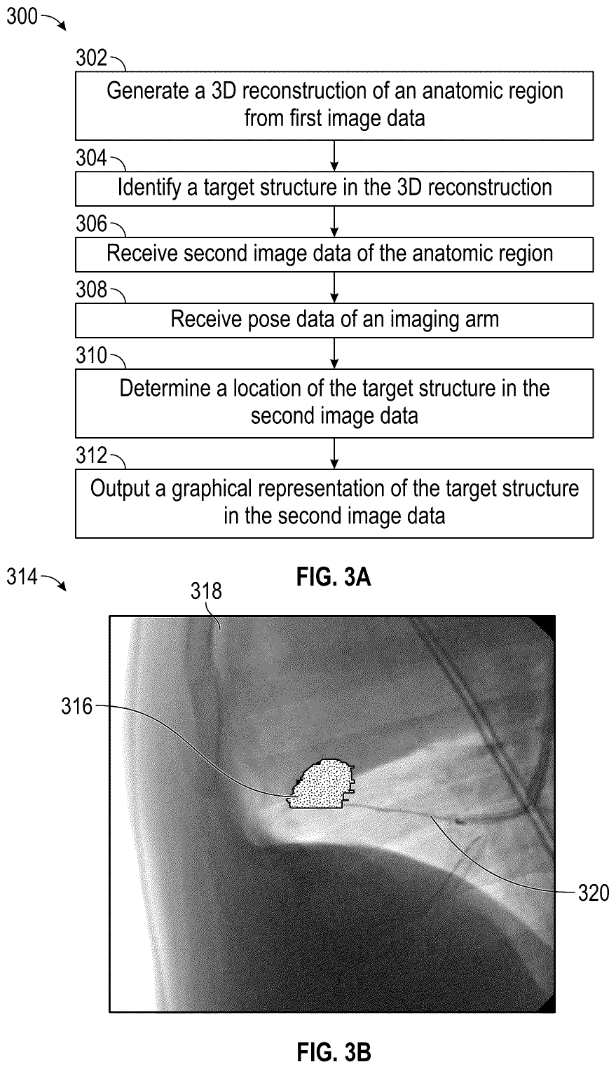

FIG. 3A is a flow diagram illustrating a method 300 for imaging an anatomic region, in accordance with embodiments of the present technology. The method 300 can be used to augment, annotate, update, or otherwise update 2D image data (e.g., fluoroscopic data or other live intraprocedural image data) with information from mrCBCT imaging. The method 300 begins at block 302 with generating a 3D reconstruction of an anatomic region from first image data. The 3D reconstruction can be a CBCT reconstruction produced using any of the manually-operated imaging apparatuses and methods described herein, such as the mrCBCT techniques discussed above with respect to FIGS. 1A-2. For example, the first image data can include a plurality of 2D projection images obtained while the imaging arm of the imaging apparatus is rotated through multiple angles, and the 3D reconstruction can be generated from the 2D projection images using a suitable image reconstruction algorithm. Optionally, the 2D projection images can be calibrated, e.g., by applying distortion correction parameters and/or geometric calibration parameters, before being used to generate the 3D reconstruction. The resulting 3D reconstruction can provide an intraprocedural representation of the patient anatomy at the time of the medical procedure. In some embodiments, the 3D reconstruction is fixed in space (e.g., has a fixed origin and coordinate system) with respect to the geometry of the overall imaging system (e.g., the relative positions of the stabilized and calibrated imaging apparatus with respect to the volume or body being imaged).

At block 304, the method 300 continues with identifying at least one target structure in the 3D reconstruction. The target structure can be a tissue, structure, feature, or other object within the anatomic region that is a site of interest for a medical procedure. For example, the target structure can be a lesion or nodule that is to be biopsied and/or ablated. The target can be identified based on input from an operator, automatically by a computing device, or suitable combinations thereof. In some embodiments, the process of block 304 includes determining a location or region of the target structure in the 3D reconstruction, e.g., by segmenting graphical elements (e.g., pixels or voxels) representing the target structure in the 3D reconstruction and/or the 2D projection images used to generate the 3D reconstruction. Segmenting can be performed manually, automatically (e.g., using computer vision algorithms and/or other image processing algorithms), or semi-automatically, in accordance with techniques known to those of skill in the art. For example, the operator can select a region of interest in one or more imaging planes (e.g., a coronal, axial, and/or sagittal imaging planes) that includes the target structure. A computing device can then automatically identify and segment the target structure from the selected region.

The output of block 304 can include a set of 3D coordinates delineating the geometry and location of the target structure. For example, the coordinates can indicate the location of one or more portions of the target structure, such as the centroid and/or boundary points. The coordinates can be identified with respect to the origin and coordinate system of the 3D reconstruction of block 302. Alternatively or in combination, the processes of block 304 can include extracting or otherwise identifying various morphological features of the target structures, such as the size, shape, boundaries, surface features, etc. A 3D model or other virtual representation of the target structure can be generated based on the coordinates and/or extracted morphological features, using techniques known to those of skill in the art. The 3D model can have the same origin and coordinate system as the 3D reconstruction.

At block 306, the method 300 can include receiving second image data of the anatomic region. The second image data can include still images and/or video images. For example, the second image data can include 2D fluoroscopic image data providing one or more real-time or near-real-time images of the anatomic region during a medical procedure. The second image data can be acquired by the same imaging apparatus used to acquire the first image data for producing the 3D reconstruction of block 302. For example, the first and second image data can both be obtained by a manually-operated mobile C-arm apparatus. In some embodiments, the first and second image data are both acquired during the same medical procedure. The imaging apparatus can remain in substantially the same position relative to the patient when acquiring both the first and second image data so that the second image data can be geometrically related to the first image data, as described in greater detail below. The imaging apparatus can be considered to be in the same position relative to the patient even if the imaging arm is rotated to different poses, as long as the rest of the imaging apparatus remains stationary relative to the patient.

At block 308, the method 300 can include receiving pose data of an imaging arm of the imaging apparatus. The pose data can represent the pose of the imaging arm (e.g., a rotational angle or a series of rotational angles) at or near the time the second image data of block 306 was acquired. The second image data can include a single image generated at a single pose of the imaging arm or can include a plurality of images generated at a plurality of different poses of the imaging arm. In some embodiments, the pose data is generated based on sensor data from one or more sensors, such as a motion sensor (e.g., an IMU). The pose data can be temporally associated with the second image data, as described above with respect to block 208 of FIG. 2.

At block 310, the method 300 continues with determining a location of the target structure in the second image data, based on the 3D reconstruction of block 302 and the pose data of block 308. This process can be performed in many different ways. In some embodiments, block 310 includes generating a 2D projection of the target structure from the 3D reconstruction, such that the location of the target structure in the 2D projection matches the location of the target structure in the second image data. The pose data of the imaging arm can provide the point of view for the 2D projection, in accordance with geometric techniques known to those of skill in the art.

For example, as previously described, the 3D reconstruction can have a fixed origin and coordinate system relative to the imaging apparatus. The pose (e.g., angle) of the imaging arm can share the same origin and coordinate system as the 3D reconstruction. Thus, if the geometry of the overall imaging system remains the same across both the first and second image data (e.g., the imaging apparatus remains in the same position relative to the patient's body), such that the position of the origin and coordinate system of the 3D reconstruction relative to the imaging apparatus is maintained, then the location of the target structure in the 3D reconstruction can be geometrically related to the location of the target structure in the second image data using the pose of the imaging arm. Specifically, the pose of the imaging arm for each second image can provide the point of view for projecting the coordinates of the target structure from the 3D reconstruction (e.g., the centroid and/or boundary points of the target structure) onto the respective second image. In some embodiments, the target structure is represented as a 3D model or other virtual representation (e.g., as discussed above in block 304), and the pose of the imaging arm is used to determine the specific orientation at which the 3D model is projected to generate a 2D image of the target structure that matches the second image data.

As another example, the location of the target structure can be determined using the first image data used to generate the 3D reconstruction. As discussed above, the 3D reconstruction can be generated from a plurality of 2D projection images acquired at different angles of the imaging arm. The method 300 can include identifying the current angle of the imaging arm using the pose data of block 308, and then retrieving the projection image that was acquired at the same angle or a similar angle. The location of the target structure in the projection image can then be determined, e.g., using the coordinates of the target structure previously identified in block 304. Optionally, if none of the projection images were obtained at an angle that is sufficiently close to the current angle of the imaging arm, the location of the target structure can be determined by interpolating or extrapolating location information from the projection image(s) obtained at the angle(s) closest to the current angle.

The location of the target structure in the projection image can then be correlated to the location of the target structure in the second image data. In some embodiments, because the same imaging apparatus, imaging apparatus position, and patient position are used to generate both the projection images (the first image data) and the second image data, the location of the target structure in the projection image is assumed to be the same or similar to the location of the target structure in the second image data. Accordingly, the coordinates of the target structure in the projection image can be directly used as the coordinates of the target structure in the second image data. In other embodiments, however, the coordinates of the target structure in the projection image can be translated, rotated, and/or otherwise modified to map to the coordinate system of the second image data.

In some embodiments, the second image data of block 306 includes images from the imaging apparatus that have not been calibrated (e.g., by applying distortion correction parameters and/or geometric calibration parameters), while the second reconstruction is generated from images that have been calibrated (e.g., as discussed above with respect to block 208 of FIG. 2). The geometry and location of the target structure in non-calibrated image data may be different than the geometry and location in the calibrated image data (and thus, the 3D reconstruction). To compensate for these differences, the method 300 can include reversing or otherwise removing the calibration applied to the 3D reconstruction and/or the first image data, before using the 3D reconstruction and/or the first image data in the processes of block 310. For example, each of the first images can be reverted to their non-calibrated state. The non-calibrated first images can be used to generate a non-calibrated 3D reconstruction of the target structure, and the non-calibrated 3D reconstruction can be used to produce 2D projections as discussed above in block 310. As another example, in embodiments where the method 300 includes generating a 3D model of the target structure from calibrated data (e.g., as discussed above in block 304), the model can be modified to reverse or otherwise remove the effects of any calibration processes on the geometry and/or location of the target structure. In some embodiments, reversing the calibration on the model includes applying one or more rigid or non-rigid transformations to the model (e.g., translation, rotation, warping) that revert any transformations resulting from the distortion correction and/or geometric calibration processes. The modified 3D model can then be projected to generate a 2D image of the target structure, as discussed above. In other embodiments, however, the second image data of block 306 can also be calibrated, e.g., using the same or similar distortion correction parameters and/or geometric calibration parameters as the 3D reconstruction. Optionally, both the 3D reconstruction and the second image data can be produced without any distortion correction and/or geometric calibration processes.

At block 312, the method 300 can include outputting a graphical representation of the target structure in the second image data. The graphical representation can include a virtual rendering of the target structure that is overlaid onto the second image data. For example, the location and geometry of a target nodule can be virtually projected onto live fluoroscopy data to provide augmented fluoroscopic images. The graphical representation can include shading, highlighting, coloring, borders, labels, arrows, and/or any other suitable visual indicator identifying the target structure in the second image data. The graphical representation can be displayed to an operator via a user interface to provide image-based guidance for various procedures, such as navigating a tool to the target structure, positioning the tool at or within the target structure, treating the target structure with the tool, etc.

FIG. 3B is a representative example of an augmented fluoroscopic image 314 that can be generated using the processes of the method 300 of FIG. 3A, in accordance with embodiments of the present technology. Specifically, the augmented fluoroscopic image 314 can be output to an operator in connection with block 312 of the method 300. The augmented fluoroscopic image 314 includes a graphical representation of a target structure 316 overlaid onto a live 2D fluoroscopic image 318. In the illustrated embodiment, the target structure 316 is depicted as a highlighted or colored region to visually distinguish the target structure 316 from the surrounding anatomy in the 2D fluoroscopic image 318. Thus, the operator can view the augmented fluoroscopic image 314 for guidance in positioning a tool 320 at or within the target structure 316.

Referring again to FIG. 3A, in some embodiments, the process of block 312 further includes updating the graphical representation to reflect changes in the imaging setup. For example, if the imaging arm is rotated to a different pose, the location of the target in the 2D images may also change. In such embodiments, the method 300 can include detecting the change in pose of the imaging arm (e.g., using the techniques described above with respect to block 308), determining the new location of the target in the second image data (e.g., as described above with respect to block 310), and modifying the graphical representation so the target is depicted at the new location in the image data.

The method 300 can provide various advantages compared to conventional augmented fluoroscopy techniques. For example, the method 300 can be performed without requiring preprocedural image data (e.g., CT scan data) to generate the 3D reconstruction. Instead, the 3D reconstruction can be generated solely from intraprocedural data, which can provide a more accurate representation of the actual anatomy. The method 300 can also utilize the same imaging apparatus to generate the 3D reconstruction and obtain live 2D images, which can simplify the overall procedure and reduce the amount of equipment needed. Additionally, the method 300 can be performed without relying on a fiducial marker board or other physical structure to provide a reference for registering the second images to the 3D reconstruction. Imaging techniques that use a fiducial marker board may be constrained to a limited rotation range since the markers in the board may not be visible at certain angles. In contrast, the present technology allows for imaging over a larger rotation range, which can improve the accuracy and image quality of the reconstruction.

The features of the method 300 shown in FIG. 3A can be modified in many different ways. For example, the processes of the method 300 can be performed in a different order than the order shown in FIG. 3A, e.g., the process of block 308 can be performed before or concurrently with the process of block 306, the process of blocks 306 and/or 308 can be performed before or concurrently with the process of blocks 302 and/or 304, etc. Additionally, some of the processes of the method 300 can be omitted in other embodiments. Although the method 300 is described above with reference to a single target structure, in other embodiments the method 300 can be performed for multiple target structures within the same anatomic region.

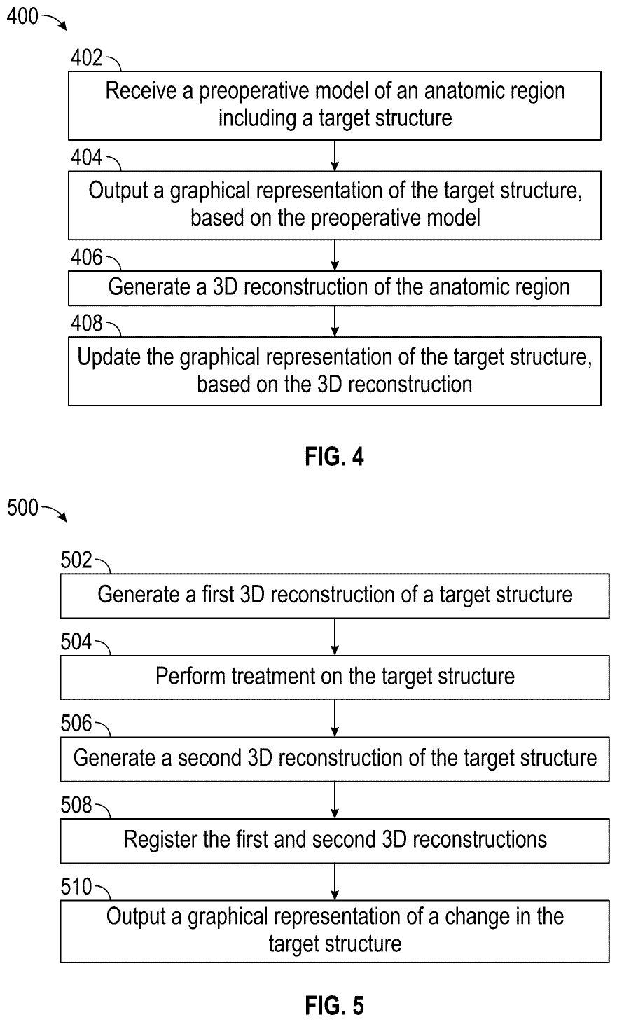

FIG. 4 is a flow diagram illustrating a method 400 for imaging an anatomic region during a medical procedure, in accordance with embodiments of the present technology. The method 400 can be used to re-register, update, or otherwise modify a preoperative model of the anatomic region using an intraprocedural CBCT reconstruction. In some situations, the preoperative model may not accurately reflect the actual state of the patient anatomy at the time of the procedure. The divergence between the actual anatomy and the preoperative model can make it difficult or impossible for the operator to navigate a tool to a desired target in the anatomic region and/or accurately apply treatment to the target. The method 400 can address these shortcomings by using intraprocedural mrCBCT to revise the preoperative model to reflect the actual patient anatomy.

The method 400 begins at block 402 with receiving a preoperative model of the anatomic region. The preoperative model can be a 2D or 3D representation of the anatomy generated from preoperative or preprocedural image data (e.g., preoperative CT scan data). The model can be generated from the preoperative data in accordance with techniques known to those of skill in the art, such as by automatically, semi-automatically, or manually segmenting the image data to generate a plurality of model components representing structures within the anatomic region (e.g., passageways, tissues, etc.). In some embodiments, the preoperative model is generated at least 12 hours, 24 hours, 48 hours, 36 hours, 72 hours, 1 week, 2 weeks, or 1 month before a medical procedure (e.g., a biopsy or treatment procedure) is performed in the anatomic region.

The preoperative model can include at least one target structure for the medical procedure, such as a lesion or nodule to be biopsied. In some embodiments, the method 400 includes determining a location of the target structure from the preoperative image data. For example, the target structure can be automatically, semi-automatically, or manually segmented from the preoperative image data in accordance with techniques known to those of skill in the art.

At block 404, the method 400 can continue with outputting a graphical representation of the target structure, based on the preoperative model. The graphical representation can be a 2D or 3D virtual rendering of the target structure and/or surrounding anatomy that is displayed to an operator to provide image-based guidance during the medical procedure. The location of the target structure can be determined from the preoperative model of block 402. For example, the graphical representation can display the preoperative model to serve as a map of the patient anatomy, and can include visual indicators (e.g., shapes, coloring, shading, etc.) marking the location of the target structure in the preoperative model.

The graphical representation can also show a location of a tool in order to assist the operator in navigating the tool to the target structure. For example, the graphical representation can include another visual indicator representing the tool, such as a virtual rendering or model of the tool, a marker showing the location of the tool relative to the target structure, etc. The graphical representation can be updated as the operator moves the tool within the anatomic region to provide real-time or near-real-time navigation guidance and feedback (e.g., via EM tracking, shape sensing, and/or image-based techniques). In such embodiments, the tool can be registered to the preoperative model using techniques known to those of skill in the art, such as EM navigation or shape sensing technologies. The registration can map the location of the tool within the anatomic region to the coordinate system of the preoperative model, thus allowing the tool to be tracked via the preoperative model.

At block 406, the method 400 includes generating a 3D reconstruction of the anatomic region. The 3D reconstruction can be generated using any of the systems, devices, and methods described herein, such as the mrCBCT techniques discussed above with respect to FIGS. 1A-2. The 3D reconstruction can be an intraoperative or intraprocedural representation of the patient anatomy, rather than a preoperative representation. Accordingly, the 3D reconstruction can provide a more accurate depiction of the actual state of the anatomy at the time of the medical procedure. The 3D reconstruction can show the target structure and, optionally, at least a portion of the tool deployed in the anatomic region.

At block 408, the method 400 continues with updating the graphical representation of the target structure, based on the 3D reconstruction. The graphical representation can initially show the location of the target structure as determined from the preoperative model, as discussed above in block 404. However, the preoperative model may not accurately depict the actual location of the target structure (e.g., CT-to-body divergence). Accordingly, intraprocedural image data from the 3D reconstruction of blocks 406 and 408 can be used to update or otherwise modify the graphical representation to show the correct location of the target structure.

In some embodiments, the process of block 408 includes determining the locations of the target structure and/or the tool in the 3D reconstruction. The process of block 408 can be generally similar to the process of block 304 of FIG. 3A. For example, the locations of the target structure and/or tool in the 3D reconstruction can be determined by manually, automatically, or semi-automatically segmenting the target structure and/or tool in the 3D reconstruction and/or the 2D projection images used to generate the 3D reconstruction, as discussed above.

Subsequently, the preoperative model can be registered to the 3D reconstruction using the locations (e.g., coordinates) of the target structure in the preoperative model and the 3D reconstruction. In some embodiments, the target structure is used as a landmark for registration because it is present in both the preoperative model and the 3D reconstruction. Alternatively or in combination, the tool can be used as a landmark for registering the 3D reconstruction to the preoperative model. The registration of preoperative model to the 3D reconstruction can be performed in accordance with local and/or landmark-based registration techniques known to those of skill in the art.

Once registered, the location of the target structure in the 3D reconstruction can be compared to the location of the target structure in the preoperative model to identify any discrepancies. For example, the tool navigation system (e.g., EM navigation system or shape sensing system) may indicate that the tip of a tool is within the target structure in the preoperative model, while the 3D reconstruction may show that the target structure is still a certain distance away from the tip of the tool. In some embodiments, if a discrepancy is detected, the 3D reconstruction is used to correct the location of the target structure in the preoperative model. In such embodiments, the updated graphical representation can display the preoperative model with the corrected target structure location so the operator can reposition the tool, if appropriate.

Alternatively, the 3D reconstruction can be used to partially or fully replace the preoperative model. For example, the portions of the preoperative model depicting the target structure and nearby anatomy can be replaced with the corresponding portions of the 3D reconstruction. In such embodiments, the method 400 can optionally include registering the tool to the 3D reconstruction (e.g., using EM navigation, shape sensing, and/or image-based techniques). Subsequently, the updated graphical representation can show the 3D reconstruction along with the tracked tool location.

The features of the method 400 shown in FIG. 4 can be modified in many different ways. For example, although the method 400 is described above with reference to a single target structure, in other embodiments the method 400 can be performed for multiple target structures within the same anatomic region. Additionally, some or all of the processes of the method 400 can be repeated. In some embodiments, the processes of blocks 406-408 are performed multiple times to generate 3D reconstructions of different portions of the anatomic region. Each of these 3D reconstructions can be used to update and/or replace the corresponding portion of the preoperative model, e.g., to provide more accurate navigation guidance at various locations within the anatomy.

FIG. 5 is a flow diagram illustrating a method 500 for imaging an anatomic region during a treatment procedure, in accordance with embodiments of the present technology. In some embodiments, the method 500 is used to monitor the progress of the treatment procedure, such as an ablation procedure. For example, an ablation procedure performed in the lung can include introducing a probe bronchoscopically into a target structure (e.g., a nodule or lesion), and ablating the tissue via microwave ablation, radiofrequency ablation, cryoablation, or any other suitable technique. The ablation procedure may require highly accurate intraoperative imaging (e.g., CBCT imaging) so that the operator knows where to place the probe. Specifically, before applying treatment, the operator may need to confirm that the probe is in the correct location (e.g., inside the target) and not too close to any critical structures (e.g., the heart). Intraoperative imaging can also be used to confirm whether the target structure has been sufficiently ablated. If the ablation coverage is insufficient, the probe can be repositioned and the ablation procedure repeated until enough target tissue has been ablated.

In some situations, it can be difficult to detect subtle changes in the target tissue from image data. To facilitate visual assessment, images of the target before ablation can be subtracted from images of the target after ablation to provide a graphical representation of the tissue that was ablated, also known as subtraction imaging. Subtraction imaging can make it easier for the operator to assess the extent and locations of unablated tissue. However, conventional techniques for subtraction imaging typically require injection of a contrast agent to enhance tissue changes in the pre- and post-ablation images. Additional, conventional techniques may use deformable registration based on the location of the contrast agent to align the pre- and post-ablation images with each other, which can lead to registration errors due to changes in tissue position between images.

These shortcomings can be addressed by the features of the method 500 described herein. For example, in some embodiments, the method 500 is performed without introducing any contrast agent into the anatomic region. This approach can be used for procedures performed in anatomic regions that naturally exhibit high contrast in image data. For example, the method 500 can be used to generate CT subtraction images of the lung since lung tissue is primarily air and therefore provides a very dark background on which subtle changes in tissue density can be seen.

The method 500 begins at block 502 with generating a first 3D reconstruction (“first reconstruction”) of a target structure in an anatomic region. The first reconstruction can be generated using any of the systems, devices, and methods described herein, such as the mrCBCT techniques discussed above with respect to FIGS. 1A-2. In some embodiments, the target structure is a tissue, lesion, nodule, etc., to be treated (e.g., ablated) during a medical procedure. The first reconstruction can be generated before any treatment has been applied to the target structure in order to provide a pre-treatment (e.g., pre-ablation) representation of the target structure.

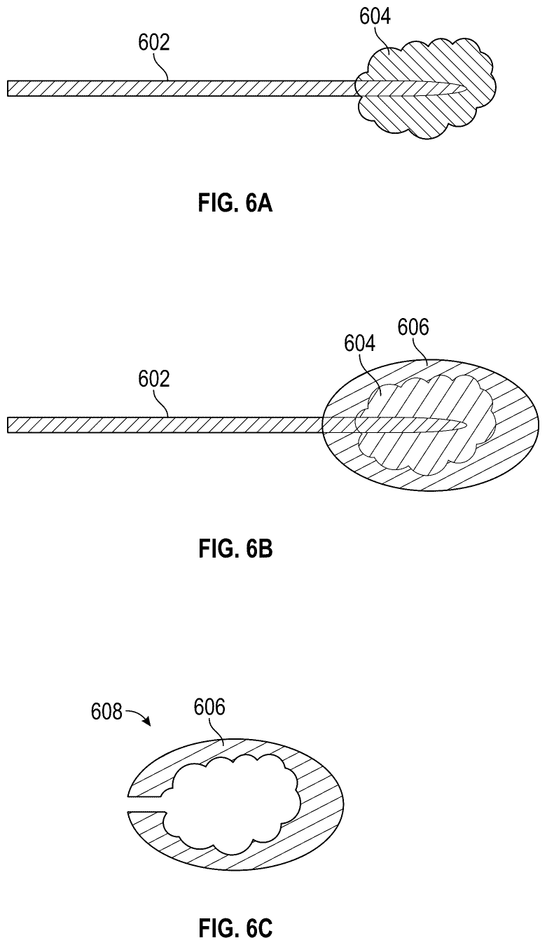

In some embodiments, the process of block 502 is performed after a tool (e.g., an ablation probe or other treatment device) has been introduced into the anatomic region and deployed to a location within or near the target structure. For example, FIG. 6A is a partially schematic illustration of a tool 602 positioned within a target structure 604, in accordance with embodiments of the present technology. The tool 602 can be positioned manually or via a robotically-controlled system, as described further below. Once the tool 602 is positioned at the desired location relative to the target structure 604, the tool 602 can be imaged along with the target structure 604 to generate the first reconstruction. Accordingly, the first reconstruction can depict at least a portion of the tool 602 together with the target structure 604. In other embodiments, however, the first reconstruction can be generated before the tool 602 is deployed.

Referring again to FIG. 5, at block 504, the method 500 continues with performing a treatment on the target structure. The treatment can include ablating, removing material from, delivering a substance to, or otherwise altering the tissue of the target structure. The treatment can be applied via a tool positioned within or near the target structure, as discussed above in block 502. For example, FIG. 6B is a partially schematic illustration of the tool 602 and target structure 604 after a treatment procedure (e.g., ablation procedure) has been applied to the target structure 604 by the tool 602. Depending on the location of the tool 602 and/or the treatment parameters (e.g., amount and/or duration of ablation energy applied), there may still be one or more regions of untreated tissue 606 within or near the target structure 604 after treatment has been applied.

Referring again to FIG. 5, at block 506, the method 500 can include generating a second 3D reconstruction (“second reconstruction”) of the target structure. The second reconstruction can be generated using any of the systems, devices, and methods described herein, such as the mrCBCT techniques discussed above with respect to FIGS. 1A-2. For example, the second reconstruction can be generated using the same techniques and imaging apparatus as the first reconstruction. The second reconstruction can be generated after the treatment process of block 504 to provide a post-treatment (e.g., post-ablation) representation of the target structure. In some embodiments, the second reconstruction is generated while the tool remains within or near the target structure, such that the second reconstruction depicts a portion of the tool together with the target structure. In other embodiments, however, the second reconstruction is generated after the tool has been removed.

At block 508, the method 500 can further include registering the first and second reconstructions to each other. The registration process can include determining a set of transformation parameters to align the first and second reconstructions to each other. The registration can be performed using any suitable rigid or non-rigid registration process or algorithm known to those of skill in the art. For example, in embodiments where the first and second reconstructions each include the treatment tool, the tool itself can be used to perform a local registration, rather than performing a global registration between the entirety of each reconstruction. This approach can be advantageous since tools are generally made of high density materials (e.g., metal) and thus can be more easily identified in the image data (e.g., CT images). Additionally, the amount of deformable motion between the target structure and the tool can be reduced or minimized because the target structure will generally be located adjacent or near the tool. Moreover, the shape of the tool is generally not expected to change in the pre-treatment versus post-treatment images, such that using the tool as the basis for local registration can improve registration accuracy and efficiency.

Accordingly, in some embodiments, the registration process of block 508 includes identifying a location of the tool in the first reconstruction, identifying a location of the tool in the second reconstruction, and registering the first and second reconstructions to each other based on the identified tool locations. The tool locations in the reconstructions can be identified using automatic, semi-automatic, or manual segmentation techniques known to those of skill in the art. Subsequently, the registration algorithm can align the tool locations in the respective reconstructions to determine the registration parameters. Optionally, the registration process can be performed on 2D image data (e.g., the 2D images used to generate the 3D reconstructions and/or 2D image slices of the 3D reconstructions), rather than the 3D reconstructions.

In embodiments where the tool is used as the basis for registration, the method 500 can include processing steps to reduce or eliminate image artifacts associated with the tool. For example, tools made partially or entirely out of metal can produce metallic image artifacts in CT images (e.g., streaks) that may obscure underlying tissue changes. Accordingly, image processing techniques such as metal artifact reduction or suppression can be applied to the 3D reconstructions and/or the 2D images used to generate the 3D reconstructions in order to mitigate image artifacts. The image processing techniques can be applied at any suitable stage in the method 500, such as before, during, or after the registration process of block 508.

At block 510, the method 500 continues with outputting a graphical representation of a change in the target structure, based on the first and second reconstructions. The graphical representation can include a 2D or 3D rendering of tissue changes in the target structure that are displayed to an operator via a graphical user interface. For example, after the reconstructions have been aligned with each other in block 508, the first reconstruction (or 2D image slices of the first reconstruction) can be subtracted or otherwise removed from the second reconstruction (or 2D image slices of the second reconstruction) to generate a subtraction image showing the remaining tissue in the target structure after treatment. As another example, the first and second reconstructions (or their respective 2D image slices) can be overlaid onto each other, displayed side-by-side, or otherwise presented together so the operator can visually assess the differences between the reconstructions.

For example, FIG. 6C is a partially schematic illustration of a subtraction image 608 generated using from pre-treatment (FIG. 6A) and post-treatment (FIG. 6B) reconstructions of the target structure 604. As shown in FIG. 6C, the image 608 shows the geometry and location of the untreated tissue 606 so the operator can visually assess the extent of treatment coverage.

Although the method 500 of FIG. 5 is described above with reference to a single target structure, in other embodiments the method 500 can be performed for multiple target structures within the same anatomic region. Additionally, in some embodiments, some or all of the processes of the method 500 can be repeated. For example, if the operator determines from the graphical representation that the target structure was not adequately treated (e.g., insufficient ablation coverage), the operator can reposition the treatment tool and then repeat some or all of the processes of the method 500 in order to apply additional treatment. This procedure can be iteratively repeated until the desired treatment has been achieved.

In some embodiments, the present technology provides methods for operating an imaging apparatus in combination with a robotic system. The robotic system can be or include any robotic assembly, manipulator, platform, etc., known to those of skill in the art for automatically or semi-automatically controlling a tool (e.g., an endoscope) within the patient's anatomy. The robotic assembly can be used to perform various medical or surgical procedures, such as a biopsy procedure, an ablation procedure, or any of the other diagnostic or treatment procedures described herein.

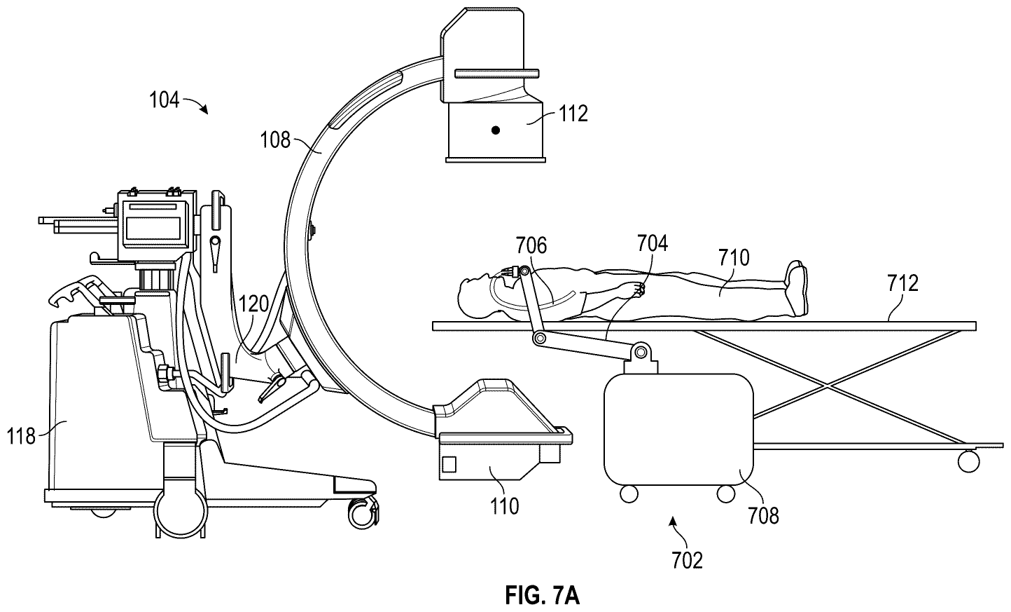

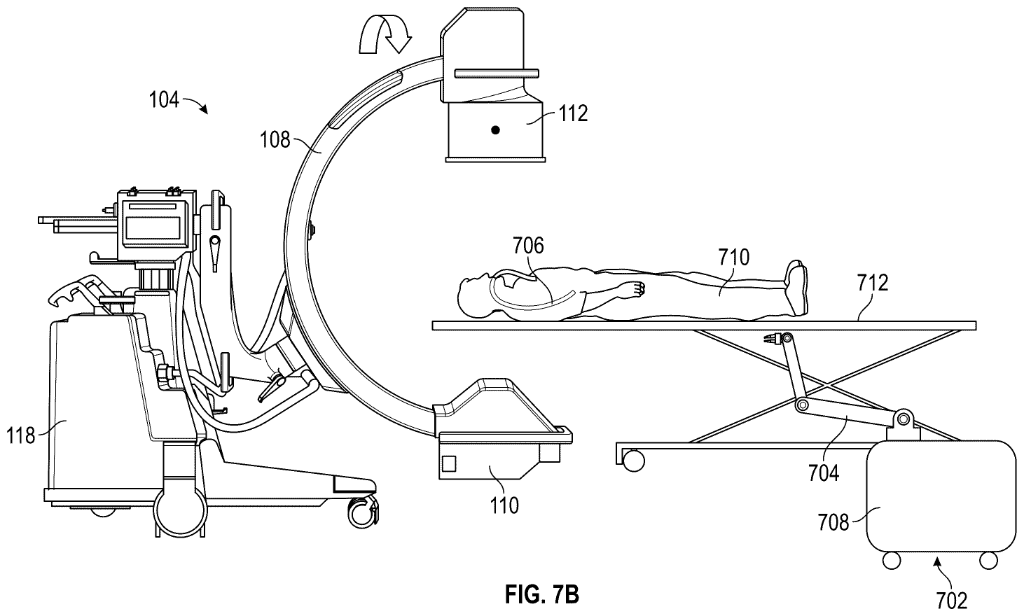

FIGS. 7A and 7B are partially schematic illustrations of the imaging apparatus 104 and a robotic assembly 702, in accordance with embodiments of the present technology. Referring first to FIG. 7A, the robotic assembly 702 includes at least one robotic arm 704 coupled to a tool 706. The robotic arm 704 can be a manipulator or similar device for supporting and controlling the tool 706, as is known to those of skill in the art. The robotic arm 704 can include various linkages, joints, actuators, etc., for adjusting the pose of the robotic arm 704 and/or tool 706. Although FIG. 7A depicts the robotic assembly 702 as including a single robotic arm 704, in other embodiments, the robotic assembly 702 can include two, three, four, five, or more robotic arms 704 that can be moved independently of each other, each controlling a respective tool. The robotic arm 704 is coupled to an assembly base 708, which can be a movable or stationary structure for supporting the robotic arms 704. The assembly base 708 can also include or be coupled to input devices (not shown) for receiving operator commands to control the robotic arm 704 and/or tool 706, such as one or more joysticks, trackballs, touchpads, keyboards, mice, etc.

During a medical procedure, the robotic assembly 702 can be positioned near a patient 710 on an operating table 712. The robotic arm 704 and/or tool 706 can be actuated, manipulated, or otherwise controlled (e.g., manually by an operator, automatically by a control system, or a combination thereof) so the tool 706 is introduced into the patient's body and positioned at a target location in the anatomy. In some embodiments, the tool 706 is registered to a model of the patient anatomy (e.g., a preoperative or intraoperative model) so the location of the tool 706 can be determined with respect to the model, e.g., for navigation purposes. Tool registration can be performed using shape sensors, EM sensors, and/or other suitable registration techniques known to those of skill in the art.

In some situations, the presence of the robotic assembly 702 limits the rotational range of the imaging apparatus 104. For example, for a bronchoscopic procedure as shown in FIG. 7A, the robotic assembly 702 can be located at or near the patient's head so the tool 706 can be introduced into the lungs via the patient's trachea. However, the imaging apparatus 104 may also need to be positioned by the patient's head in order to perform mrCBCT imaging of the lungs. As a result, the robotic assembly 702 may partially or completely obstruct the rotation of the imaging arm 108 (e.g., when a propeller rotation is performed).

Referring next to FIG. 7B, in some embodiments, the interference between the robotic assembly 702 and the imaging apparatus 104 is resolved by moving the robotic assembly 702 away from the patient 710 during imaging. For example, the once the tool 706 has been positioned at the desired location in the patient's body, the tool 706 can be disconnected (e.g., mechanically and electrically decoupled) from the robotic arm 704. The robotic arm 704 and assembly base 708 can then be moved away from the patient's body, with the tool 706 remaining in place within the patient 710. The imaging arm 108 can then be rotated through the desired angular range to generate a 3D reconstruction of the anatomy, as discussed elsewhere herein. After the imaging procedure, the assembly base 708 can be repositioned by the patient's body and the robotic arm 704 reconnected (e.g., mechanically and electrically coupled) to the tool 706.

In some embodiments, when the tool 706 is disconnected from the rest of the robotic assembly 702, the registration of the tool 706 is lost, such that the tool 706 can no longer be localized to the anatomic model. Accordingly, the present technology can provide various methods for addressing the loss of registration to provide continued tracking of the tool 706 with respect to the anatomy.

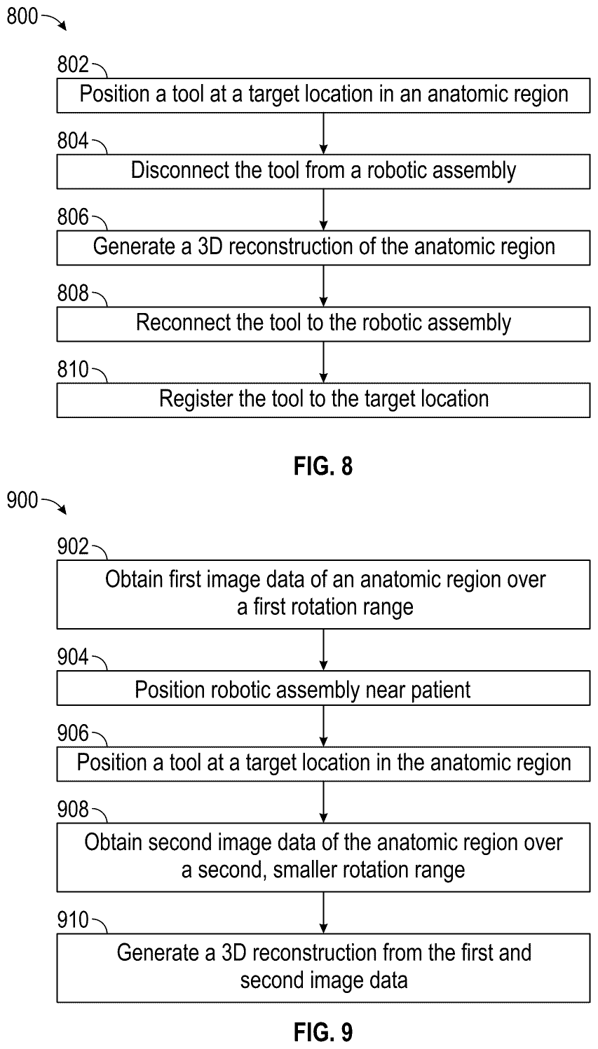

FIG. 8 is a flow diagram illustrating a method 800 for imaging an anatomic region in combination with a robotic assembly, in accordance with embodiments of the present technology. The method 800 can be used to recover the registration of a tool (e.g., the tool 706 of the robotic assembly 702 of FIGS. 7A and 7B) after the tool has been temporarily disconnected from the robotic assembly.

The method 800 begins at block 802 with positioning a tool at a target location in an anatomic region. The target location can be a location within or near a target structure, such as a nodule or lesion to be biopsied or treated. In some embodiments, the tool is positioned by a robotic assembly, e.g., automatically, based on control signals from the operator, or suitable combinations thereof. The process of block 802 can include using a model of the anatomic region to track the location of the tool and navigate the tool to the location of a target structure. The tool can be registered to the model as discussed elsewhere herein.

At block 804, the method 800 continues with disconnecting the tool from the robotic assembly. The tool can be mechanically and electrically separated from the rest of the robotic assembly (e.g., from the robotic arm supporting tool) so the robotic assembly can be moved away from the patient. When disconnected, the tool can remain at its last position within the anatomic structure, but may go limp (e.g., to reduce the risk of injury to the patient). As discussed above, the tool may lose its registration with the model when decoupled from the robotic assembly.

At block 806, the method 800 can include generating a 3D reconstruction of the anatomic region. The 3D reconstruction can be generated using any of the systems, devices, and methods described herein, such as the mrCBCT techniques discussed above with respect to FIGS. 1A-2. For example, the 3D reconstruction can be generated from 2D images acquired during a manual rotation (e.g., a manual propeller rotation) of an imaging arm of a mobile C-arm apparatus or other manually-operated imaging apparatus. In some embodiments, because the robotic assembly has been moved away from the patient, the imaging arm can be rotated through a larger rotational range, e.g., a rotational range of at least at least 90°, 100°, 110°, 120°, 130°, 140°, 150°, 160°, 170°, 180°, 190°, 200°, 210°, 220°, 230°, 240°, 250°, 260°, 270°, 280°, 290°, 300°, 310°, 320°, 330°, 340°, 350°, or 360°.

In some embodiments, block 806 further includes outputting a graphical representation to the operator, based on the 3D reconstruction. The graphical representation can show the target location in the anatomy together with at least a portion of the tool. Accordingly, the operator can view to the graphical representation to confirm whether the tool is positioned appropriately relative to the target location, e.g., for biopsy, ablation, or other purposes.

At block 808, the method 800 can include reconnecting the tool to the robotic assembly. Once the imaging of block 806 has been completed, the robotic assembly can be moved back to its original location near the patient. The tool can then be mechanically and electrically coupled to the robotic assembly so the robotic assembly can be used to control the tool. For example, if the operator determines that the tool should be adjusted (e.g., based on the 3D reconstruction of block 806), the operator may need to reconnect the tool to the robotic assembly in order to reposition the tool.

At block 810, the method 800 can optionally include registering the tool to the target location in the anatomic region. As described above, when the tool is disconnected in block 804, the original registration between the tool and the anatomic model may be lost. The registration process of block 810 can thus be used to recover the previous registration and/or generate a new registration for tracking the tool within the anatomy. For example, in some embodiments, the previous registration and/or location of the tool can be saved before disconnecting the tool in block 804. When the tool is reconnected in block 808, the previous registration and/or tool location can be reapplied. Accordingly, the pose of the tool with respect to the target location can be recovered.

[01.20] As another example, the method 800 can include using the 3D reconstruction of block 806 to generate a new registration for the tool. This approach can involve processing the 3D reconstruction to identify the locations of the target structure and the tool in the reconstructed data. In some embodiments, the target structure and tool are segmented from the 3D reconstruction or from 2D image slices of the 3D reconstruction. The segmentation can be performed using any suitable technique known to those of skill in the art, as discussed elsewhere herein. The locations of the target structure and tool can then be used to determine the pose of the tool relative to the target structure. For example, the tool pose can be expressed in terms of distance and orientation of the tool tip with respect to the target structure.

The tool can then be registered to the target location by correlating the tool pose to actual pose measurements of the tool (e.g., pose measurements generated by a shape sensor or EM tracker). In some embodiments, the tool is registered to the target location in the 3D reconstruction. The registration can allow the tool to be tracked relative to the 3D reconstruction, so that the 3D reconstruction can be used to provide image-based guidance for navigating the tool (e.g., with known tracking techniques such as EM tracking, shape sensing, and/or image based approaches). In other embodiments, however, the tool can instead be re-registered to the target location in the initial model of block 802.

Once the tool registration is complete, the operator can reposition the tool relative to the target, if desired. For example, if the operator determines that the tool was not positioned properly after viewing the 3D reconstruction generated in block 806, the operator can navigate the tool to a new location. The processes of blocks 804-810 can then be repeated to disconnect the tool from the robotic assembly, perform mrCBCT imaging of the new tool location, and reconnect and re-register the tool to the robotic assembly. This procedure can be repeated until the desired tool placement has been achieved. Additionally, although the method 800 of FIG. 8 is described above with reference to a single target location, in other embodiments, the method 800 can be repeated to perform mrCBCT imaging of multiple target locations within the same anatomic region.

In some embodiments, the mrCBCT techniques described herein are performed without repositioning the robotic assembly. Instead, the imaging arm can be rotated to a smaller angular range to avoid interfering with the robotic assembly. In such embodiments, the imaging apparatus can include sensors and/or other electronics to monitor the rotational position of the imaging arm and, optionally, alert the operator when the imaging arm is nearing or exceeding the permissible rotation range.

Alternatively or in combination, the imaging apparatus can include a stop mechanism that constrains the rotation of the imaging arm to a predetermined range, e.g., to prevent the operator from inadvertently colliding with the robotic assembly during manual rotation. The stop mechanism can be a mechanical device that physically prevents the imaging arm from being rotated past the safe range. The stop mechanism can be configured in many different ways. For example, the stop mechanism can include a clamp device which reversibly or permanently attaches to the imaging arm and/or the support arm (e.g., to the proximal portion 124 of the support arm 120 near the second interface 128 with the base 118, as shown in FIG. 1A). The stop mechanism can include at least one elongate arm extending outward from the clamp device. The operator can adjust the position of the arm to place it in the rotation path of the support arm and/or imaging arm to physically obstruct the support arm and/or imaging arm from rotating beyond a certain angular range. Alternatively or in combination, the support arm and/or imaging arm can be coupled to a tether (e.g., a rope, adjustable band, etc.) that is connected to a stationary location (e.g., on the base 118 of FIG. 1A or other location in the operating environment). The tether can be configured so that as the support arm and/or imaging arm reaches the boundary of the permissible rotation range, the tether tightens and prevents further rotation. In a further example, the stop mechanism can be a protective cover or barrier (e.g., a solid dome of a lightweight, strong material such as plexiglass) that is placed over the robotic assembly or a portion thereof (e.g., the robotic arm) to prevent contact with the imaging arm and/or support arm.

FIG. 9 is a flow diagram illustrating a method 900 for imaging an anatomic region, in accordance with embodiments of the present technology. The method 900 can be used in situations where the imaging arm is rotated to a limited angular range to accommodate a robotic assembly (e.g., the robotic assembly 702 of FIG. 7A). In some situations, the image data acquired over the limited range may not produce a 3D reconstruction with sufficient quality for confirming tool placement and/or other applications where high accuracy is important. The method 900 can address this shortcoming by supplementing the limited rotation image data with image data obtained over a larger rotation range.

The method 900 begins at block 902 with obtaining first image data of the anatomic region over a first rotation range. The first image data can be obtained using any of the systems, devices, and methods described herein, such as the mrCBCT techniques discussed above with respect to FIGS. 1A-2. In some embodiments, the first image data is acquired before the robotic assembly is positioned near the patient. Accordingly, the imaging arm can be rotated through a larger rotation range (e.g., the maximum range), such as a rotation range of at least 90°, 100°, 110°, 120°, 130°, 140°, 150°, 160°, 170°, 180°, 190°, 200°, 210°, 220°, 230°, 240°, 250°, 260°, 270°, 280°, 290°, 300°, 310°, 320°, 330°, 340°, 350°, or 360°.

Optionally, the method 900 can include generating an initial 3D reconstruction from the first image data. The 3D reconstruction can depict one or more target structures within the anatomic region, such as a nodule or lesion to be biopsied, treated, etc. The target structure can be segmented from the 3D reconstruction using any of the techniques described herein. In some embodiments, the initial 3D reconstruction depicts the anatomic region before any tool or instrument has been introduced into the patient's body.

At block 904, the method 900 can continue with positioning a robotic assembly near the patient. As previously discussed, the robotic assembly can be positioned at any suitable location that allows a tool to be introduced into the patient's body via the robotic assembly. For example, for a bronchoscopic procedure, the robotic assembly can be positioned near the patient's head. In some embodiments, the robotic assembly is moved into place while the imaging apparatus remains at the same location used to generate the first reconstruction. Optionally, the imaging apparatus can be moved to a different location to accommodate the robotic assembly.

At block 906, the method 900 can optionally include positioning a tool at a target location in the anatomic region. The target location can be a location within or near the target structure. The tool can be positioned by the robotic assembly, e.g., automatically, based on control signals from the operator, or suitable combinations thereof, as discussed elsewhere herein. In some embodiments, the tool is registered to the initial 3D reconstruction generated from the first image data of block 902, e.g., using any suitable technique known to those of skill in the art. The initial 3D reconstruction can be displayed to the operator to provide image guidance for navigating the tool to the target location, as discussed elsewhere herein.

At block 908, the method 900 continues with obtaining second image data of the anatomic region over a second, smaller rotation range. The second image data can be obtained using any of the systems, devices, and methods described herein, such as the mrCBCT techniques discussed above with respect to FIGS. 1A-2. For example, the second image data can be acquired using the same imaging apparatus that was used to acquire the first image data in block 902. In some embodiments, the second image data is acquired after the robotic assembly is positioned near the patient, such that the rotational movement of the imaging arm is limited by the presence of the robotic assembly. Accordingly, the second rotation range can be smaller than the first rotation range, such as at least 10°, 20°, 30°, 40°, 50°, 60°, 70°, 80°, 90°, 100°, 110°, 120°, 130°, 140°, 150°, 160°, 170°, or 180° smaller.

At block 910, the method 900 can include generating a 3D reconstruction from the first and second image data. In some embodiments, because the second rotation range is smaller than the first rotation range, a 3D reconstruction generated from the second image data alone may not be sufficiently accurate. Accordingly, the first image data can be combined with or otherwise used to supplement the second image data to improve the accuracy and quality of the resulting 3D reconstruction. In some embodiments, the first image data provides extrapolated and/or interpolated images at angular positions that are missing from the second image data. The resulting 3D reconstruction can thus be a “hybrid” reconstruction generated from both the first and second image data. For example, if the first image data was acquired with a 160° rotation and the second image data was acquired with a 110° rotation, the images acquired in the 50° rotation missing from the second image data can be added to the second image data. Thus, the 3D reconstruction can be generated from images spanning the full 160° rotation range, which can improve the image quality of the reconstruction.

The method 900 can optionally include outputting a graphical representation of the 3D reconstruction to an operator. The graphical representation can show the position of the tool relative to the target location, as discussed elsewhere herein. Accordingly, the operator can view the graphical representation to determine whether the tool has been placed properly. If desired, the processes of blocks 906-910 can be repeated to reposition the tool and perform mrCBCT imaging to confirm the new tool location. At least some or all of the processes of the method 900 can be performed multiple times to position the tool at multiple target locations.

Additionally, although some embodiments of the method 900 are described herein in connection with positioning a tool with a robotic assembly, the method 900 can also be used in other applications where the rotation of the imaging apparatus is constrained, e.g., due to the presence of other equipment, the location of the patient's body, etc. In such embodiments, the processes of blocks 904 and/or 906 are optional and may be omitted.

In certain situations, it may be difficult to properly align the field of view of the imaging apparatus with the target structure before the tool has been deployed, e.g., during block 902 of the method 900. Because the field of view of the CBCT reconstruction is smaller than the field of view of the projection images, the target structure may need to be at or near the center of the projection images to ensure that it will also be visible in the reconstruction. In a conventional imaging procedure, the tip portion of the tool can be used as a target for aligning the imaging apparatus with the target. However, this would not be possible for an initial mrCBCT reconstruction performed before the tool and robotic assembly are in place.

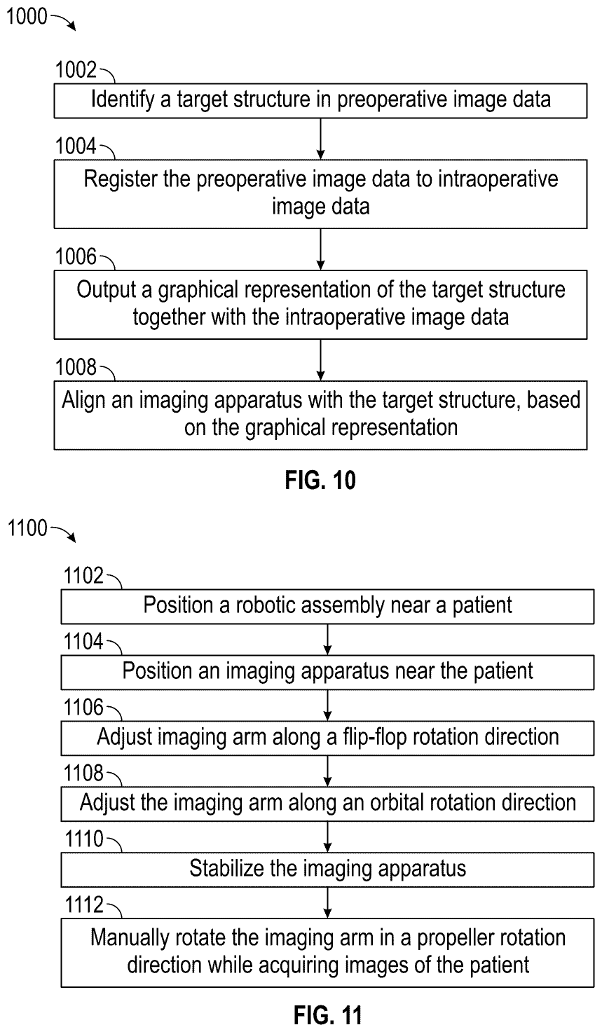

FIG. 10 is a flow diagram illustrating a method 1000 for aligning an imaging apparatus with a target structure, in accordance with embodiments of the present technology. The method 1000 can be used to align the field of view of the imaging apparatus without relying on an internally-positioned tool as the reference. Accordingly, the method 1000 can be performed before and/or during the process of block 902 of the method 900 of FIG. 9 to ensure that the target structure will be visible in the initial 3D reconstruction.

The method 1000 begins at block 1002 with identifying a target structure in preoperative image data. The target structure can be a lesion, nodule, or other object of interest in an anatomic region of a patient. The preoperative image data can include preoperative CT scan data or any other suitable image data of the patient's anatomy obtained before a medical procedure is performed on the patient. In some embodiments, the preoperative image data is generated at least 12 hours, 24 hours, 48 hours, 36 hours, 72 hours, 1 week, 2 weeks, or 1 month before the medical procedure. The preoperative image data can be provided as a 3D representation or model, as 2D images, or both. The target structure can be identified by segmenting the preoperative image data in accordance with techniques known to those of skill in the art, as described elsewhere herein.

At block 1004, the method 1000 can include registering the preoperative image data to intraoperative image data. The intraoperative image data can include still and/or video images (e.g., fluoroscopic images), and can be acquired using any suitable imaging apparatus, such as any of the systems and devices described herein. The intraoperative image data can provide a real-time or near-real-time depiction of the current field of view of the imaging apparatus. As discussed above, the intraoperative image data can be acquired before a tool has been positioned near the target structure in the anatomy.

The registration process of block 1004 can be performed in many different ways. For example, in some embodiments, the target structure is segmented in the preoperative image data, as discussed above in connection with block 1002. The preoperative image data can then be used to generate one or more simulated 2D images that represent how the target structure would appear in the field of view of the imaging apparatus. The simulated images can be registered to the intraoperative image data, e.g., using features or landmarks of the target structure and/or of other anatomic structures visible in both the simulated images and the intraoperative image data, in accordance with landmark-based registration techniques known to those of skill in the art. For example, for a bronchoscopic procedure, the landmarks for registration can include the patient's ribs, spine, and/or heart.

At block 1006, the method 1000 continues with outputting a graphical representation of the target structure together with the intraoperative image data. The graphical representation can include, for example, a 2D or 3D rendering of the target structure overlaid onto the intraoperative image data, e.g., similar to the graphical representation of block 312 of FIG. 3A. The location of the target structure in the intraoperative image data can be determined using the registration of block 1004. The method 1000 can also include updating the graphical representation as the imaging setup is changed (e.g., as the operator moves the imaging apparatus, rotates the imaging arm, etc.), as discussed above in block 312 of FIG. 3A.

At block 1008, the method 1000 further includes aligning the imaging apparatus with the target structure, based on the graphical representation of block 1006. For example, the operator can adjust the imaging apparatus (e.g., rotate the imaging arm) so that the target structure is at or near the center of the intraoperative image data. The alignment can optionally be performed in multiple imaging planes (e.g., frontal and lateral imaging planes) to increase the likelihood of the target structure being visible in the image reconstruction. Once the imaging apparatus has been aligned, the imaging apparatus can then be used to perform mrCBCT imaging of the target structure, as described elsewhere herein.

FIG. 11 is a flow diagram illustrating a method 1100 for using an imaging apparatus in combination with a robotic assembly, in accordance with embodiments of the present technology. The method 1100 can be performed with a manually-operated imaging apparatus (e.g., the imaging apparatus 104 of FIG. 1A). The method 1100 can allow the mrCBCT techniques described herein to be performed in combination with a robotic assembly (e.g., the robotic assembly 702 of FIGS. 7A and 7B). As discussed above, the presence of the robotic assembly may constrain the rotational range of the imaging apparatus. The method 1100 can be used to adjust the setup of the imaging apparatus to accommodate the robotic assembly while also maintaining the ability to rotate the imaging arm over a relatively large angular range.

The method 1100 begins at block 1102 with positioning a robotic assembly near a patient. The robotic assembly can be or include any robotic system, manipulator, platform, etc., known to those of skill in the art for automatically or semi-automatically controlling a tool within the patient's anatomy. The robotic assembly can be used to perform various medical or surgical procedures, such as a biopsy procedure, an ablation procedure, or other suitable diagnostic or treatment procedure. The robotic assembly can deploy the tool into the patient's body and navigate the tool to a target anatomic location (e.g., a lesion to be biopsied, ablated, treated, etc.).

At block 1104, the method 1100 can continue with positioning an imaging apparatus (e.g., the imaging apparatus 104 of FIG. 1A) near the patient. The imaging apparatus can be used to acquire images of the patient's anatomy to confirm whether the tool is positioned at the desired location. However, the presence of the robotic assembly near the patient may interfere with the rotation (e.g., propeller and/or orbital rotation) of the imaging arm of the imaging apparatus. For example, in a bronchoscopic procedure, the robotic assembly can be positioned near the patient's head so the tool can be deployed into the patient's airways via the trachea. The imaging apparatus can also be positioned near the patient's head in order to acquire images of the patient's chest region.

At block 1106, the method 1100 can include adjusting the imaging arm along a flip-flop rotation direction. As discussed above with respect to FIG. 1D, a flip-flop rotation can include rotating the imaging arm and the distal portion of the support arm relative to the remaining portion of the support arm and the base of the imaging apparatus. Adjusting the imaging arm along the flip-flop rotation direction can reposition the imaging arm relative to the robotic assembly so that the imaging arm can subsequently perform a propeller rotation over a large angular range (e.g., a range of at least 90°, 120°, 150°, 180°, 210°, 240°, 270°, 300°, or 330°) without colliding with the robotic assembly. In some embodiments, the adjustment includes rotating the imaging arm along the flip-flop rotation direction by at least 10°, 20°, 30°, 40°, 50°, 60°, 70°, 80°, or 90° (e.g., from a starting position of 0° of flip-flop rotation). Optionally, the imaging apparatus can include markers or other visual indicators that guide the operator in manually adjusting the imaging arm to the appropriate flip-flop rotational position. Once the desired positioning is achieved, the imaging arm can be locked to prevent further flip-flop rotation.

At block 1108, the method 1100 can optionally include adjusting the imaging arm along an orbital rotation direction. In some embodiments, the flip-flop rotation in block 1106 causes the detector of the imaging apparatus to become misaligned with the propeller rotation axis of the imaging apparatus and/or the patient's body (e.g., the surface of the detector is at an angle relative to the propeller rotation axis and/or the vertical axis of the body), which may impair image quality. Accordingly, the imaging arm can be adjusted along the orbital rotation direction to realign the detector, such that the surface of the detector is substantially parallel to the propeller rotation axis and/or the vertical axis of the body. In some embodiments, the adjustment includes rotating the imaging arm along the orbital direction by 5°, 10°, 15°, 20°, 25°, 30°, 35°, 40°, or 45° (e.g., from a starting position of 0° of orbital rotation). Optionally, the imaging apparatus can include markers or other visual indicators that guide the operator in manually adjusting the imaging arm to the appropriate orbital rotational position. Once the desired positioning is achieved, the imaging arm can be locked to prevent further orbital rotation. In other embodiments, however, block 1108 is optional and can be omitted altogether.

At block 1110, the method 1100 can include stabilizing the imaging apparatus. The stabilization process can be performed using any of the techniques described herein, such as by using one or more shim structures. In some embodiments, the stabilization process is performed after the flip-flop and/or orbital adjustments have been made because the shim structures can inhibit certain movements of the imaging arm (e.g., orbital rotation).

At block 1112, the method 1100 continues with manually rotating the imaging arm in a propeller rotation direction while acquiring images of the patient. In some embodiments, the imaging arm is able to rotate over a larger range of angles without contacting the robotic assembly, e.g., compared to an imaging arm that has not undergone the flip-flop and/or orbital adjustments described above. For example, the imaging arm can be rotated in the propeller rotation direction over a range of at least 90°, 120°, 150°, 180°, 210°, 240°, 270°, 300°, or 330°. The images acquired during the propeller rotation can be used to generate a 3D reconstruction of the patient anatomy, as described elsewhere herein. The 3D reconstruction can then be used to verify whether the tool is positioned at the desired location in the patient's body.

Some embodiments of the methods described herein involve identifying a location of a tool from a 3D reconstruction. With mrCBCT imaging, if the rotation range is less than 180° and/or if there are subtle misalignments of the 2D projection images, then a tool within the 3D reconstruction generated from the 2D projection images can sometimes appear blurred and/or with significant artifacts. These phenomena can prevent identification of the precise location of the tool relative to surrounding structures (e.g., the tip of a biopsy needle can appear unfocused). This can lead to challenges in identifying location of the tool relative to a target structure, e.g., it may be difficult to determine if a tip of a biopsy needle is within a lesion or on the edge of it. To aid in the identification of the location of a tool (or other structure) within a 3D reconstruction, one technique includes identifying the location of the tool (or a portion thereof, such as the tool tip) in one or more of the 2D projection images (e.g., automatically, semi-automatically, or manually). This identification can then be used to determine the tool location in the 3D reconstruction, e.g., via triangulation or other suitable techniques. Subsequently, a graphical representation of the tool location can be overlaid onto or otherwise displayed with the 3D reconstruction (e.g., a colored line can represent a biopsy needle, a dot can represent the needle tip). Optionally, if the tool location cannot be determined with sufficient certainty (e.g., the triangulation of the identified tool locations in the 2D projection images do not align precisely within the 3D reconstruction), then the graphical representation can include a colored region or similar visual indicator showing the probability distribution for the tool location. The center of the region can represent the most likely true location of the tool, and the probability of the tool being at a particular location in the region can decrease with increased distance from the center. The approaches described herein can provide the operator with a clearer visual representation of the location of the tool (or portion thereof) with respect to the surrounding anatomic structures.

### Examples

The following examples are included to further describe some aspects of the present technology, and should not be used to limit the scope of the technology.
1. A system for imaging an anatomic region, the system comprising:
one or more processors;
a display; and a memory operably coupled to the one or more processors and storing instructions that, when executed by the one or more processors, cause the system to perform operations comprising:
   generating a 3D reconstruction of an anatomic region from first image data obtained using an imaging apparatus;
   identifying a target structure in the 3D reconstruction;
   receiving second image data of the anatomic region obtained using the imaging apparatus;
   receiving pose data of an imaging arm of the imaging apparatus; and
   outputting, via the display, a graphical representation of the target structure overlaid onto the second image data, based on the pose data and the 3D reconstruction.
2. The system of Example 1, wherein generating the 3D reconstruction comprises:
receiving a plurality of projection images from the imaging apparatus while the imaging arm is manually rotated;
determining pose information of the imaging arm for each projection image; and generating the 3D reconstruction based on the projection images and the pose information.
3. The system of Example 2, further comprising a shim structure configured to stabilize the imaging arm during the manual rotation.
4. The system of Example 2 or Example 3, wherein the manual rotation comprises a rotation of at least 90 degrees.
5. The system of any one of Examples 2-4, wherein the operations further comprise: determining a current pose of the imaging arm, based on the pose data;
identifying a projection image that was acquired at the same or a similar pose as the current pose; and
determining a location of the target structure in the second image data, based on the identified projection image.
6. The system of Example 5, wherein the location of the target structure in the second image data corresponds to a location of the target structure in the identified target image.
7. The system of any one of Examples 2-4, wherein the operations further comprise:
generating a 3D model of the target structure;
determining a current pose of the imaging arm, based on the pose data; and
generating a 2D projection of the 3D model from a point of view corresponding to the current pose of the imaging arm; and
determining a location of the target structure in the second image data, based on the 2D projection.
8. The method of any one of Examples 5-7, wherein the pose data is generated using sensor data from at least one sensor coupled to the imaging arm.
9. The method of Example 8, wherein the at least one sensor comprises a motion sensor.
10. The method of Example 9, wherein the motion sensor comprises an inertial measurement unit (IMU).
11. The system of any one of Examples 1-10, wherein the 3D reconstruction is generated during a medical procedure performed on the patient and the second image data is generated during the same medical procedure.
12. The system of any one of Examples 1-11, wherein the 3D reconstruction is generated without using preoperative image data of the anatomic region.
13. The system of any one of Examples 1-12, wherein identifying the target structure includes segmenting the target structure in the 3D reconstruction.
14. The system of any one of Examples 1-13, wherein the 3D reconstruction comprises a CBCT image reconstruction and the second image data comprises live fluoroscopic images of the anatomic region.
15. The system of any one of Examples 1-14, wherein the operations further comprise updating the graphical representation after the imaging arm is rotated to a different pose.
16. The system of any one of Examples 1-15, wherein the operations further comprise calibrating the first image data before generating the 3D reconstruction.
17. The system of Example 16, wherein calibrating the first image data includes one or more of (a) applying distortion correction parameters to the first image data or (b) applying geometric calibration parameters to the first image data.
18. The system of Example 16 or Example 17, wherein the operations further comprise reversing calibration of a 3D model of the target structure generated from the calibrated first image data, before using the 3D model to determine a projected location of the target structure in the second image data.
19. A method for imaging an anatomic region of a patient, the method comprising:
generating a 3D representation of an anatomic region using first images acquired by an imaging apparatus;
identifying a target location in the 3D representation;
receiving a second image of the anatomic region from the imaging apparatus;
determining a pose of the imaging arm of the imaging apparatus associated with the second image; and
displaying an indicator of the target location together with the second image, based on the determined pose and the 3D representation.
20. A non-transitory computer-readable storage medium comprising instructions that, when executed by one or more processors of a computing system, cause the computing system to perform operations comprising:
generating a 3D reconstruction of an anatomic region using first image data from an imaging apparatus;
identifying a target structure in the 3D reconstruction;
receiving second image data of the anatomic region from the imaging apparatus;
receiving pose data of an imaging arm of the imaging apparatus; and
determining a location of the target structure in the second image data, based on the pose data and the 3D reconstruction.
21. A system for imaging an anatomic region, the system comprising:
one or more processors; and
a memory operably coupled to the one or more processors and storing instructions that, when executed by the one or more processors, cause the system to perform operations comprising:
   receiving a preoperative model of the anatomic region;
   outputting a graphical representation of a target structure in the anatomic region, based on the preoperative model;
   generating a 3D reconstruction of the anatomic region using an imaging apparatus; and
   updating the graphical representation of the target structure in the anatomic region, based on the 3D reconstruction.
22. The system of Example 21, wherein generating the 3D reconstruction comprises:
receiving a plurality of 2D images from the imaging apparatus while manually rotating an imaging arm of the imaging apparatus;
determining pose information of the imaging arm for each 2D image; and
generating the 3D reconstruction based on the 2D images and the pose information.
23. The system of Example 22, further comprising a shim structure configured to stabilize the imaging arm during manual rotation.
24. The system of Example 22 or Example 23, wherein the manual rotation comprises a rotation of at least 90 degrees.
25. The system of any one of Examples 22-24, wherein generating the 3D reconstruction comprises calibrating the 2D images by one or more of (a) applying distortion correction parameters to the 2D images or (b) applying geometric calibration parameters to the 2D images.
26. The system of any one of Examples 21-25, wherein the 3D reconstruction is generated during a medical procedure performed on the patient and the preoperative model is generated before the medical procedure.
27. The system of any one of Examples 21-26, wherein the 3D reconstruction is generated independently of the preoperative model.
28. The system of any one of Examples 21-27, wherein updating the graphical representation comprises:
comparing a location of the target structure in the preoperative model to a location of the target structure in the 3D reconstruction; and
modifying the graphical representation to show the target structure at the location in the 3D reconstruction.
29. The system of any one of Examples 21-28, wherein the graphical representation shows a location of a tool relative to the target structure.
30. A method for imaging an anatomic region during a medical procedure, the method comprising:
outputting a graphical representation of a target structure in the anatomic region, wherein a location of the target structure in the graphical representation is determined based on preoperative image data;
generating a 3D representation of the anatomic region during the medical procedure; and
modifying the graphical representation of the target structure, wherein a location of the target structure in the modified graphical representation is determined based on the 3D representation.
31. A non-transitory computer-readable storage medium comprising instructions that, when executed by one or more processors of a computing system, cause the computing system to perform operations comprising:
determining a location of a target structure in a preoperative model of an anatomic region;
outputting a graphical representation of the target structure, based on the determined location of the target structure in the preoperative model;
generating a 3D reconstruction of the anatomic region using an imaging apparatus;
determining a location of the target structure in the 3D reconstruction; and
updating the graphical representation of the target structure, based on the determined location of the target structure in the 3D reconstruction.
32. A system for imaging an anatomic region, the system comprising:
one or more processors; and
a memory operably coupled to the one or more processors and storing instructions that, when executed by the one or more processors, cause the system to perform operations comprising:
   generating a first 3D reconstruction of a target structure in the anatomic region using an imaging apparatus;
   after a treatment has been applied to the target structure, generating a second 3D reconstruction of the target structure using the imaging apparatus; and
   outputting a graphical representation showing a change in the target structure after the treatment, based on the first and second 3D reconstructions.
33. The system of Example 32, wherein the first and second 3D reconstructions are each generated by:
receiving a plurality of 2D images from the imaging apparatus while manually rotating an imaging arm of the imaging apparatus;
determining pose information of the imaging arm for each 2D image; and
generating the 3D reconstruction based on the 2D images and the pose information.
34. The system of Example 33, further comprising a shim structure configured to stabilize the imaging arm during the manual rotation.
35. The system of Example 33 or Example 34, wherein the manual rotation comprises a rotation of at least 90 degrees.
36. The system of any one of Examples 32-35, wherein the treatment comprises ablating at least a portion of the target structure.
37. The system of Example 36, wherein the graphical representation shows a remaining portion of the target structure after the ablation.
38. The system of any one of Examples 32-37, wherein the graphical representation comprises a subtraction image generated between the first and second 3D reconstructions.
39. The system of any one of Examples 32-38, wherein the operations further comprise registering the first 3D reconstruction to the second 3D reconstruction.
40. The system of Example 39, wherein the first and second 3D reconstructions are registered based on a location of a tool in the first and second 3D reconstructions.
41. The system of Example 39 or Example 40, wherein the first and second 3D reconstructions are registered using a rigid registration process.
42. A method for imaging an anatomic region, the method comprising:
generating a first 3D representation of a target structure in the anatomic region; after a treatment has been applied to the target structure, generating a second 3D representation of the target structure;
determining a change in the target structure after the treatment based on the first and second 3D representations; and outputting a graphical representation of the change.
43. A non-transitory computer-readable storage medium comprising instructions that, when executed by one or more processors of a computing system, cause the computing system to perform operations comprising:
generating a first 3D reconstruction of a target structure in the anatomic region;
receiving an indication that a treatment has been applied to the target structure;
generating a second 3D reconstruction of the target structure after the treatment; and
determining a change in the target structure after the treatment, based on the first and second 3D reconstructions.
44. A system for imaging an anatomic region, the system comprising:
a robotic assembly configured to navigate a tool within the anatomic region;
one or more processors operably coupled to the robotic assembly; and
a memory operably coupled to the one or more processors and storing instructions that, when executed by the one or more processors, cause the system to perform operations comprising:
   receiving signals causing the robotic assembly to position the tool at a target location in the anatomic region;
   receiving a first indication that the tool has been disconnected from the robotic assembly;
   generating a 3D reconstruction of the anatomic region while the tool is disconnected from the robotic assembly, using an imaging apparatus;
   receiving a second indication that the tool has been reconnected to the robotic assembly; and registering the tool to the target location.
45. The system of Example 44, wherein the 3D reconstruction is generated by:
receiving a plurality of 2D images from the imaging apparatus while manually rotating an imaging arm of the imaging apparatus;
determining pose information of the imaging arm for each 2D image; and
generating the 3D reconstruction based on the 2D images and the pose information.
46. The system of Example 45, further comprising a shim structure configured to stabilize the imaging arm during the manual rotation.
47. The system of Example 45 or Example 46, wherein the manual rotation comprises a rotation of at least 90 degrees.
48. The system of any one of Examples 44-47, wherein the tool comprises an endoscope.
49. The system of any one of Examples 44-48, wherein the operations further comprise registering the tool to a preoperative model of the anatomic region, before disconnecting the tool from the robotic assembly.
50. The system of Example 49, wherein the tool is registered to the target location by applying a saved registration between the tool and the preoperative model.
51. The system of Example 49, wherein the tool is registered to the target location by generating a new registration for the tool, based on a pose of the tool in the 3D reconstruction.
52. The system of Example 51, wherein the new registration comprises (1) a registration between the tool and the 3D reconstruction or (2) a registration between the tool and the preoperative model.
53. The system of any one of Examples 44-52, wherein the operations further comprise tracking a location of the tool within the anatomic region, based on the registration.
54. A method for imaging an anatomic region, the method comprising:
navigating, via a robotic assembly, a tool to a target structure in the anatomic region;
disconnecting the tool from the robotic assembly;
generating, via an imaging apparatus, a 3D reconstruction of the anatomic region while the tool is disconnected from the robotic assembly;
reconnecting the tool to the robotic assembly; and
registering the tool to the anatomic region from the 3D reconstruction.
55. A non-transitory computer-readable storage medium comprising instructions that, when executed by one or more processors of a computing system, cause the computing system to perform operations comprising:
receiving signals causing a robotic assembly to position a tool at a target location in an anatomic region;
after the tool has been disconnected from the robotic assembly, generating a 3D reconstruction of the anatomic region using an imaging apparatus; and
after the tool has been reconnected to the robotic assembly, registering the tool to the target location.
56. A system for imaging an anatomic region using an imaging apparatus, the system comprising:
one or more processors; and
a memory operably coupled to the one or more processors and storing instructions that, when executed by the one or more processors, cause the system to perform operations comprising:
obtaining first image data of the anatomic region while an imaging arm of the imaging apparatus is rotated over a first rotation range;
obtaining second image data of the anatomic region while the imaging arm is rotated over a second rotation range, the second rotation range being smaller than the first rotation range; and
generating a 3D reconstruction of the anatomic region from the first and second image data.
57. The system of Example 56, wherein the operations further comprise:
determining pose information of the imaging arm for each image in the first and second image data; and
generating the 3D reconstruction from the first and second image data and the pose information.
58. The system of Example 56 or Example 57, wherein the first rotation range is at least 90 degrees.
59. The system of any one of Examples 56-58, wherein the 3D reconstruction is generated by combining the first and second image data.
60. The system of Example 59, wherein combining the first and second image data comprises adding at least one image from the first image data to the second image data, wherein the at least one image is obtained while the imaging arm is at a rotational angle outside the second rotation range.
61. The system of any one of Examples 56-60, further comprising a stop mechanism configured to constrain rotation of the imaging arm to a predetermined range.
62. The system of any one of Examples 56-61, further comprising a robotic assembly configured to control a tool within the anatomic region.
63. The system of Example 62, wherein the first image data is obtained while the robotic assembly is spaced apart from the imaging apparatus, and the second image data is obtained while the robotic assembly is near the imaging apparatus.
64. The system of Example 62 or Example 63, wherein the 3D reconstruction depicts a portion of the tool within the anatomic region.
65. The system of any one of Examples 56-64, wherein the operations further comprise aligning a field of view of the imaging apparatus with a target structure in the anatomic region, before obtaining the first image data.
66. The system of Example 65, wherein the field of view is aligned by:
identifying the target structure in preoperative image data of the anatomic region;
registering the preoperative image data to intraoperative image data generated by the imaging apparatus;
outputting a graphical representation of the target structure overlaid onto the imaging apparatus, based on the registration; and aligning the field of view based on the graphical representation.
67. A method for imaging an anatomic region of a patient using an imaging apparatus, the method comprising:
obtaining first image data of the anatomic region while an imaging arm of the imaging apparatus is rotated over a first rotation range;
positioning a robotic assembly near the patient;
obtaining second image data of the anatomic region while the imaging arm is rotated over a second rotation range, the second rotation range being smaller than the first rotation range; and generating a 3D reconstruction of the anatomic region from the first and second image data.
68. A non-transitory computer-readable storage medium comprising instructions that, when executed by one or more processors of a computing system, cause the computing system to perform operations comprising:
obtaining first image data of the anatomic region while an imaging arm of an imaging apparatus is rotated over a first rotation range;
obtaining second image data of the anatomic region while the imaging arm is rotated over a second rotation range, the second rotation range being smaller than the first rotation range;
modifying the second image data by adding at least one image from the first image data; and
generating a 3D reconstruction from the modified second image data.

### CONCLUSION

Although many of the embodiments are described above with respect to systems, devices, and methods for performing a medical procedure in a patient's lungs, the technology is applicable to other applications and/or other approaches, such as medical procedures performed in other anatomic regions (e.g., the musculoskeletal system). Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1A-11.

The various processes described herein can be partially or fully implemented using program code including instructions executable by one or more processors of a computing system for implementing specific logical functions or steps in the process. The program code can be stored on any type of computer-readable medium, such as a storage device including a disk or hard drive. Computer-readable media containing code, or portions of code, can include any appropriate media known in the art, such as non-transitory computer-readable storage media. Computer-readable media can include volatile and non-volatile, removable and non-removable media implemented in any method or technology for storage and/or transmission of information, including, but not limited to, random-access memory (RAM), read-only memory (ROM), electrically erasable programmable read-only memory (EEPROM), flash memory, or other memory technology; compact disc read-only memory (CD-ROM), digital video disc (DVD), or other optical storage; magnetic cassettes, magnetic tape, magnetic disk storage, or other magnetic storage devices; solid state drives (SSD) or other solid state storage devices; or any other medium which can be used to store the desired information and which can be accessed by a system device.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

As used herein, the terms “generally,” “substantially,” “about,” and similar terms are used as terms of approximation and not as terms of degree, and are intended to account for the inherent variations in measured or calculated values that would be recognized by those of ordinary skill in the art.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. As used herein, the phrase “and/or” as in “A and/or B” refers to A alone, B alone, and A and B.

To the extent any materials incorporated herein by reference conflict with the present disclosure, the present disclosure controls.

It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

What is claimed is:

1. A system for imaging an anatomic region, the system comprising:
one or more processors;
a display; and
a memory operably coupled to the one or more processors and storing instructions that, when executed by the one or more processors, cause the system to perform operations comprising:
generating a 3D reconstruction of an anatomic region from first image data obtained using an imaging apparatus;
identifying a target structure in the 3D reconstruction;
receiving second image data of the anatomic region obtained using the imaging apparatus;
receiving pose data of an imaging arm of the imaging apparatus; and
outputting, via the display, a graphical representation of the target structure overlaid onto the second image data, based on the pose data and the 3D reconstruction.

2. The system of claim 1, wherein generating the 3D reconstruction comprises:
receiving a plurality of projection images from the imaging apparatus while the imaging arm is manually rotated;
determining pose information of the imaging arm for each projection image; and
generating the 3D reconstruction based on the projection images and the pose information.

3. The system of claim 2, further comprising a shim structure configured to stabilize the imaging arm during the manual rotation.

4. The system of claim 2 or claim 3, wherein the manual rotation comprises a rotation of at least 90 degrees.

5. The system of any one of claims 2-4, wherein the operations further comprise:
determining a current pose of the imaging arm, based on the pose data;
identifying a projection image that was acquired at the same or a similar pose as the current pose; and
determining a location of the target structure in the second image data, based on the identified projection image.

6. The system of claim 5, wherein the location of the target structure in the second image data corresponds to a location of the target structure in the identified target image.

7. The system of any one of claims 2-4, wherein the operations further comprise:
generating a 3D model of the target structure;
determining a current pose of the imaging arm, based on the pose data; and
generating a 2D projection of the 3D model from a point of view corresponding to the current pose of the imaging arm; and
determining a location of the target structure in the second image data, based on the 2D projection.

8. The method of any one of claims 5-7, wherein the pose data is generated using sensor data from at least one sensor coupled to the imaging arm.

9. The method of claim 8, wherein the at least one sensor comprises a motion sensor.

10. The method of claim 9, wherein the motion sensor comprises an inertial measurement unit (IMU).

11. The system of any one of claims 1-10, wherein the 3D reconstruction is generated during a medical procedure performed on the patient and the second image data is generated during the same medical procedure.

12. The system of any one of claims 1-11, wherein the 3D reconstruction is generated without using preoperative image data of the anatomic region.

13. The system of any one of claims 1-12, wherein identifying the target structure includes segmenting the target structure in the 3D reconstruction.

14. The system of any one of claims 1-13, wherein the 3D reconstruction comprises a CBCT image reconstruction and the second image data comprises live fluoroscopic images of the anatomic region.

15. The system of any one of claims 1-14, wherein the operations further comprise updating the graphical representation after the imaging arm is rotated to a different pose.

16. The system of any one of claims 1-15, wherein the operations further comprise calibrating the first image data before generating the 3D reconstruction.

17. The system of claim 16, wherein calibrating the first image data includes one or more of (a) applying distortion correction parameters to the first image data or (b) applying geometric calibration parameters to the first image data.

18. The system of claim 16 or claim 17, wherein the operations further comprise reversing calibration of a 3D model of the target structure generated from the calibrated first image data, before using the 3D model to determine a projected location of the target structure in the second image data.

19. A method for imaging an anatomic region of a patient, the method comprising:
generating a 3D representation of an anatomic region using first images acquired by an imaging apparatus;
identifying a target location in the 3D representation;
receiving a second image of the anatomic region from the imaging apparatus;
determining a pose of the imaging arm of the imaging apparatus associated with the second image; and
displaying an indicator of the target location together with the second image, based on the determined pose and the 3D representation.

20. A non-transitory computer-readable storage medium comprising instructions that, when executed by one or more processors of a computing system, cause the computing system to perform operations comprising:
generating a 3D reconstruction of an anatomic region using first image data from an imaging apparatus;
identifying a target structure in the 3D reconstruction;
receiving second image data of the anatomic region from the imaging apparatus;
receiving pose data of an imaging arm of the imaging apparatus; and
determining a location of the target structure in the second image data, based on the pose data and the 3D reconstruction.

21. A system for imaging an anatomic region, the system comprising:
one or more processors; and
a memory operably coupled to the one or more processors and storing instructions that, when executed by the one or more processors, cause the system to perform operations comprising:
receiving a preoperative model of the anatomic region;
outputting a graphical representation of a target structure in the anatomic region, based on the preoperative model;
generating a 3D reconstruction of the anatomic region using an imaging apparatus; and
updating the graphical representation of the target structure in the anatomic region, based on the 3D reconstruction.

22. The system of claim 21, wherein generating the 3D reconstruction comprises:
receiving a plurality of 2D images from the imaging apparatus while manually rotating an imaging arm of the imaging apparatus;
determining pose information of the imaging arm for each 2D image; and
generating the 3D reconstruction based on the 2D images and the pose information.

23. The system of claim 22, further comprising a shim structure configured to stabilize the imaging arm during manual rotation.

24. The system of claim 22 or claim 23, wherein the manual rotation comprises a rotation of at least 90 degrees.

25. The system of any one of claims 22-24, wherein generating the 3D reconstruction comprises calibrating the 2D images by one or more of (a) applying distortion correction parameters to the 2D images or (b) applying geometric calibration parameters to the 2D images.

26. The system of any one of claims 21-25, wherein the 3D reconstruction is generated during a medical procedure performed on the patient and the preoperative model is generated before the medical procedure.

27. The system of any one of claims 21-26, wherein the 3D reconstruction is generated independently of the preoperative model.

28. The system of any one of claims 21-27, wherein updating the graphical representation comprises:
comparing a location of the target structure in the preoperative model to a location of the target structure in the 3D reconstruction; and
modifying the graphical representation to show the target structure at the location in the 3D reconstruction.

29. The system of any one of claims 21-28, wherein the graphical representation shows a location of a tool relative to the target structure.

30. A method for imaging an anatomic region during a medical procedure, the method comprising:
outputting a graphical representation of a target structure in the anatomic region, wherein a location of the target structure in the graphical representation is determined based on preoperative image data;
generating a 3D representation of the anatomic region during the medical procedure; and
modifying the graphical representation of the target structure, wherein a location of the target structure in the modified graphical representation is determined based on the 3D representation.

31. A non-transitory computer-readable storage medium comprising instructions that, when executed by one or more processors of a computing system, cause the computing system to perform operations comprising:
determining a location of a target structure in a preoperative model of an anatomic region;
outputting a graphical representation of the target structure, based on the determined location of the target structure in the preoperative model;
generating a 3D reconstruction of the anatomic region using an imaging apparatus;
determining a location of the target structure in the 3D reconstruction; and
updating the graphical representation of the target structure, based on the determined location of the target structure in the 3D reconstruction.

32. A system for imaging an anatomic region, the system comprising:
one or more processors; and
a memory operably coupled to the one or more processors and storing instructions that, when executed by the one or more processors, cause the system to perform operations comprising:
generating a first 3D reconstruction of a target structure in the anatomic region using an imaging apparatus;
after a treatment has been applied to the target structure, generating a second 3D reconstruction of the target structure using the imaging apparatus; and
outputting a graphical representation showing a change in the target structure after the treatment, based on the first and second 3D reconstructions.

33. The system of claim 32, wherein the first and second 3D reconstructions are each generated by:
receiving a plurality of 2D images from the imaging apparatus while manually rotating an imaging arm of the imaging apparatus;
determining pose information of the imaging arm for each 2D image; and
generating the 3D reconstruction based on the 2D images and the pose information.

34. The system of claim 33, further comprising a shim structure configured to stabilize the imaging arm during the manual rotation.

35. The system of claim 33 or claim 34, wherein the manual rotation comprises a rotation of at least 90 degrees.

36. The system of any one of claims 32-35, wherein the treatment comprises ablating at least a portion of the target structure.

37. The system of claim 36, wherein the graphical representation shows a remaining portion of the target structure after the ablation.

38. The system of any one of claims 32-37, wherein the graphical representation comprises a subtraction image generated between the first and second 3D reconstructions.

39. The system of any one of claims 32-38, wherein the operations further comprise registering the first 3D reconstruction to the second 3D reconstruction.

40. The system of claim 39, wherein the first and second 3D reconstructions are registered based on a location of a tool in the first and second 3D reconstructions.

41. The system of claim 39 or claim 40, wherein the first and second 3D reconstructions are registered using a rigid registration process.

42. A method for imaging an anatomic region, the method comprising:
generating a first 3D representation of a target structure in the anatomic region;
after a treatment has been applied to the target structure, generating a second 3D representation of the target structure;
determining a change in the target structure after the treatment based on the first and second 3D representations; and
outputting a graphical representation of the change.

43. A non-transitory computer-readable storage medium comprising instructions that, when executed by one or more processors of a computing system, cause the computing system to perform operations comprising:
generating a first 3D reconstruction of a target structure in the anatomic region;
receiving an indication that a treatment has been applied to the target structure;
generating a second 3D reconstruction of the target structure after the treatment; and
determining a change in the target structure after the treatment, based on the first and second 3D reconstructions.

44. A system for imaging an anatomic region, the system comprising:
a robotic assembly configured to navigate a tool within the anatomic region;
one or more processors operably coupled to the robotic assembly; and
a memory operably coupled to the one or more processors and storing instructions that, when executed by the one or more processors, cause the system to perform operations comprising:
receiving signals causing the robotic assembly to position the tool at a target location in the anatomic region;
receiving a first indication that the tool has been disconnected from the robotic assembly;
generating a 3D reconstruction of the anatomic region while the tool is disconnected from the robotic assembly, using an imaging apparatus;
receiving a second indication that the tool has been reconnected to the robotic assembly; and
registering the tool to the target location.

45. The system of claim 44, wherein the 3D reconstruction is generated by:
receiving a plurality of 2D images from the imaging apparatus while manually rotating an imaging arm of the imaging apparatus;
determining pose information of the imaging arm for each 2D image; and
generating the 3D reconstruction based on the 2D images and the pose information.

46. The system of claim 45, further comprising a shim structure configured to stabilize the imaging arm during the manual rotation.

47. The system of claim 45 or claim 46, wherein the manual rotation comprises a rotation of at least 90 degrees.

48. The system of any one of claims 44-47, wherein the tool comprises an endoscope.

49. The system of any one of claims 44-48, wherein the operations further comprise registering the tool to a preoperative model of the anatomic region, before disconnecting the tool from the robotic assembly.

50. The system of claim 49, wherein the tool is registered to the target location by applying a saved registration between the tool and the preoperative model.

51. The system of claim 49, wherein the tool is registered to the target location by generating a new registration for the tool, based on a pose of the tool in the 3D reconstruction.

52. The system of claim 51, wherein the new registration comprises (1) a registration between the tool and the 3D reconstruction or (2) a registration between the tool and the preoperative model.

53. The system of any one of claims 44-52, wherein the operations further comprise tracking a location of the tool within the anatomic region, based on the registration.

54. A method for imaging an anatomic region, the method comprising:
navigating, via a robotic assembly, a tool to a target structure in the anatomic region;
disconnecting the tool from the robotic assembly;
generating, via an imaging apparatus, a 3D reconstruction of the anatomic region while the tool is disconnected from the robotic assembly;
reconnecting the tool to the robotic assembly; and
registering the tool to the anatomic region from the 3D reconstruction.

55. A non-transitory computer-readable storage medium comprising instructions that, when executed by one or more processors of a computing system, cause the computing system to perform operations comprising:
receiving signals causing a robotic assembly to position a tool at a target location in an anatomic region;
after the tool has been disconnected from the robotic assembly, generating a 3D reconstruction of the anatomic region using an imaging apparatus; and
after the tool has been reconnected to the robotic assembly, registering the tool to the target location.

56. A system for imaging an anatomic region using an imaging apparatus, the system comprising:
one or more processors; and
a memory operably coupled to the one or more processors and storing instructions that, when executed by the one or more processors, cause the system to perform operations comprising:
obtaining first image data of the anatomic region while an imaging arm of the imaging apparatus is rotated over a first rotation range;
obtaining second image data of the anatomic region while the imaging arm is rotated over a second rotation range, the second rotation range being smaller than the first rotation range; and
generating a 3D reconstruction of the anatomic region from the first and second image data.

57. The system of claim 56, wherein the operations further comprise:
determining pose information of the imaging arm for each image in the first and second image data; and
generating the 3D reconstruction from the first and second image data and the pose information.

58. The system of claim 56 or claim 57, wherein the first rotation range is at least 90 degrees.

59. The system of any one of claims 56-58, wherein the 3D reconstruction is generated by combining the first and second image data.

60. The system of claim 59, wherein combining the first and second image data comprises adding at least one image from the first image data to the second image data, wherein the at least one image is obtained while the imaging arm is at a rotational angle outside the second rotation range.

61. The system of any one of claims 56-60, further comprising a stop mechanism configured to constrain rotation of the imaging arm to a predetermined range.

62. The system of any one of claims 56-61, further comprising a robotic assembly configured to control a tool within the anatomic region.

63. The system of claim 62, wherein the first image data is obtained while the robotic assembly is spaced apart from the imaging apparatus, and the second image data is obtained while the robotic assembly is near the imaging apparatus.

64. The system of claim 62 or claim 63, wherein the 3D reconstruction depicts a portion of the tool within the anatomic region.

65. The system of any one of claims 56-64, wherein the operations further comprise aligning a field of view of the imaging apparatus with a target structure in the anatomic region, before obtaining the first image data.

66. The system of claim 65, wherein the field of view is aligned by:
identifying the target structure in preoperative image data of the anatomic region;
registering the preoperative image data to intraoperative image data generated by the imaging apparatus;
outputting a graphical representation of the target structure overlaid onto the imaging apparatus, based on the registration; and
aligning the field of view based on the graphical representation.

67. A method for imaging an anatomic region of a patient using an imaging apparatus, the method comprising:
obtaining first image data of the anatomic region while an imaging arm of the imaging apparatus is rotated over a first rotation range;
positioning a robotic assembly near the patient;
obtaining second image data of the anatomic region while the imaging arm is rotated over a second rotation range, the second rotation range being smaller than the first rotation range; and
generating a 3D reconstruction of the anatomic region from the first and second image data.

68. A non-transitory computer-readable storage medium comprising instructions that, when executed by one or more processors of a computing system, cause the computing system to perform operations comprising:
obtaining first image data of the anatomic region while an imaging arm of an imaging apparatus is rotated over a first rotation range;
obtaining second image data of the anatomic region while the imaging arm is rotated over a second rotation range, the second rotation range being smaller than the first rotation range;
modifying the second image data by adding at least one image from the first image data; and
generating a 3D reconstruction from the modified second image data.
